(19) United States
(12) Patent Application Publication
(10) Pub. No.: US 2020/0391202 A1
(43) Pub. Date: Dec. 17, 2020

(54) Infection Detection Systems and Methods

(71) Applicant: TELEFLEX MEDICAL INCORPORATED, Morrisville, NC (US)

(72) Inventors: John C. VICTOR, Kunkletown, PA (US); David T. ROWE, Fleetwood, PA (US); Rodney W. DENLINGER, Lancaster, PA (US); Victoria E. WAGNER, Rochester Hills, MI (US); Michael A. BOUCHARD, Wyomissing, PA (US); Zheng ZHANG, Cambridge, MA (US)

(21) Appl. No.: 17/009,253

(22) Filed: Sep. 1, 2020

Related U.S. Application Data
(63) Continuation of application No. PCT/US2019/020336, filed on Mar. 1, 2019.
(60) Provisional application No. 62/773,607, filed on Nov. 30, 2018.
(60) Provisional application No. 62/637,767, filed on Mar. 2, 2018.

(51) Int. Cl. B01L 3/00; C12Q 1/6888

(52) U.S. Cl. CPC B01L 3/502; A61B 5/150992; C12Q 1/6888

(57) ABSTRACT

Systems and methods for detecting an infection are provided. A method of detecting an infection in a patient includes subcutaneously collecting a whole blood sample in a reservoir of the sampling device. The method further includes removing the reservoir, connecting the reservoir to a sample processor, and transferring the whole blood sample from the reservoir to the sample processor for processing. The method also includes connecting the sample processor to an analytical instrument and analyzing the processed sample via the analytical instrument.

### CROSS-REFERENCE TO RELATED APPLICATION

This application is a continuation of International PCT Application no. PCT/US2019/020336, filed Mar. 1, 2019, and published as WO 2019/169287 A1, and claims priority to U.S. Provisional Patent Application No. 62/637,767, filed Mar. 2, 2018, and claims priority to U.S. Provisional Patent Application No. 62/773,607, filed Nov. 30, 2018, the disclosures of which are incorporated by reference herein in their entireties.

### TECHNICAL FIELD

This application relates generally to systems, devices, and methods for collecting a sample and for processing the sample in an infection detection system.

### BACKGROUND

Patient exposure to health care environments elevates risk of systemic and/or local infections caused by commensal microorganisms. Infection risk is particularly elevated during the use of external communicating medical devices. There is accordingly a need for systems, devices, and methods for rapidly collecting, identifying, quantifying, and characterizing causative microorganisms associated with infections.

### SUMMARY

The present inventors recognize that there is a need to improve one or more features of infection detection systems and methods. For example, an infection detection system in accordance with the one aspect of the present invention includes a sampling device, a sample processor, and an analytical instrument that rapidly collects, identifies, quantifies, and characterizes causative microorganisms associated with infections. The devices and methods disclosed herein are directed to mitigating or overcoming one or more of the problems set forth above and/or other problems of the prior art.

An aspect of the various embodiments of the invention is directed to a sampling device configured to collect a sample from a medical device and to transfer the sample to a sample processor for processing of the sample. The sampling device includes an outer shell having an interior and a first open end. The first open end of the outer shell includes a connector that is configured to selectively seal the first open end to the sample processor and to the medical device. The sampling device further includes a sample collector at least partially disposed within the interior of the outer shell that is configured to collect the sample from the medical device. The connector is configured to removably connect the sample collector to the medical device to collect the sample and to removably connect the sample collector to the sample processor to transfer the sample to the sample processor. The sample collector includes a first end that is a removable witness to an interior of the medical device to collect the sample from the interior of the medical device. The sample collector is configured to extend and retract along a longitudinal axis of the outer shell. The sampling device further includes an isolation housing that is disposed within the interior of the outer shell. The isolation housing comprising a first end disposed at the first open end of the outer shell, a second end disposed opposite the first end with respect to the longitudinal axis of the outer shell, and an interior provided between the first and second ends of the isolation housing. The sample collector is at least partially provided within the interior of the isolation housing. The first end of the isolation housing includes an opening that is closed by a protective barrier that seals the interior of the isolation housing from exposure to the sample. The first end of the sample collector is configured to rupture the protective barrier upon an extension of the sample collector through the opening of the first end of the isolation housing to selectively expose the first end of the sample collector to the sample. The second end of the isolation housing includes an opening and the sample collector is slideably provided within the opening. The sample collector includes a seal disposed between an outer surface of the sample collector and an inner surface of the isolation housing to prevent the sample from passing through the opening of the second end of the isolation housing. The sampling device further comprising a guard movably connected to the outer shell, the guard being movable along a longitudinal axis of the outer shell between a stowed position in which the guard is retracted inwardly away from the first open end of the outer shell and an extended position in which the guard extends outwardly beyond the first open end of the outer shell to protect the sample collector. The guard includes a spring that biases the guard towards the extended position. The sample collector includes a surface that is configured to bind pathogens thereto. An interior of the medical device is comprised of a material, and the surface of the sample collector is comprised of the material that comprises the interior of the medical device. The connector is configured to removably connect the sample collector to the medical device such that the surface of the sample collector is tangential to adjacent interior surfaces of the medical device and such that the surface of the sample collector and the adjacent interior surfaces of the medical device together provide a continuous interior surface within the interior of medical device. The surface includes an absorbent material. The surface is configured to limit absorption of substances other than pathogens. The surface includes at least one of pores, through holes, and structured surfaces that increase an effective surface area of the surface. The connector is directly connected to an access port of the medical device. The connector comprises at least one of threads, tapers, and snaps. The medical device is at least one of a catheter, a scope, and a line.

Another aspect of the various embodiments of the invention includes a tool configured to transfer a sampling device. The sampling device is configured to collect a sample from a medical device and to transfer the sample to a sample processor for processing of the sample. The sampling device includes an outer shell having an interior and a first open end. The first open end of the outer shell includes a connector that is configured to selectively seal the first open end to the sample processor and to the medical device. The sampling device further includes a sample collector at least partially disposed within the interior of the outer shell that is configured to collect the sample from the medical device. The connector is configured to removably connect the sample collector to the medical device to collect the sample and to removably connect the sample collector to the sample processor to transfer the sample to the sample processor. The sample collector includes a first end that is a removable witness to an interior of the medical device to collect the sample from the interior of the medical device. The sample collector is configured to extend and retract along a longitudinal axis of the outer shell. The sampling device further includes an isolation housing that is disposed within the interior of the outer shell. The isolation housing comprising a first end disposed at the first open end of the outer shell, a second end disposed opposite the first end with respect to the longitudinal axis of the outer shell, and an interior provided between the first and second ends of the isolation housing. The sample collector is at least partially provided within the interior of the isolation housing. The first end of the isolation housing includes an opening that is closed by a protective barrier that seals the interior of the isolation housing from exposure to the sample. The first end of the sample collector is configured to rupture the protective barrier upon an extension of the sample collector through the opening of the first end of the isolation housing to selectively expose the first end of the sample collector to the sample. The second end of the isolation housing includes an opening and the sample collector is slideably provided within the opening. The sample collector includes a seal disposed between an outer surface of the sample collector and an inner surface of the isolation housing to prevent the sample from passing through the opening of the second end of the isolation housing. The sampling device further comprising a guard movably connected to the outer shell, the guard being movable along a longitudinal axis of the outer shell between a stowed position in which the guard is retracted inwardly away from the first open end of the outer shell and an extended position in which the guard extends outwardly beyond the first open end of the outer shell to protect the sample collector. The guard includes a spring that biases the guard towards the extended position. The sample collector includes a surface that is configured to bind pathogens thereto. An interior of the medical device is comprised of a material, and the surface of the sample collector is comprised of the material that comprises the interior of the medical device. The connector is configured to removably connect the sample collector to the medical device such that the surface of the sample collector is tangential to adjacent interior surfaces of the medical device and such that the surface of the sample collector and the adjacent interior surfaces of the medical device together provide a continuous interior surface within the interior of medical device. The surface includes an absorbent material. The surface is configured to limit absorption of substances other than pathogens. The surface includes at least one of pores, through holes, and structured surfaces that increase an effective surface area of the surface. The connector is directly connected to an access port of the medical device. The connector comprises at least one of threads, tapers, and snaps. The medical device is at least one of a catheter, a scope, and a line. The tool comprises an elongate body having a first end with a connector that is configured to selectively hold the outer shell of the sampling device to permit removal and transfer of the sampling device. The connector of the first end of the tool comprises at least one of threads, tapers, and snaps. The first end further comprises a guard movably connected to the elongate body, the guard being movable along a longitudinal axis of the elongate body between a stowed position in which the guard is retracted inwardly away from the first end of the tool and an extended position in which the guard extends outwardly beyond the first end of the tool to protect the sampling device held by the tool. The guard includes a spring that biases the guard towards the extended position. The elongate body further comprises a second end with a connector that is configured to selectively hold the outer shell of the sampling device to permit removal and transfer of the sampling device. The connector of the second end of the tool comprises at least one of threads, tapers, and snaps. The second end further comprises a guard movably connected to the elongate body. The guard is movable along the longitudinal axis of the elongate body between a stowed position in which the guard is retracted inwardly away from the second end of the tool, and an extended position in which the guard extends outwardly beyond the second end of the tool to protect the sampling device held by the second end of the tool. The guard of the second end of the tool includes a spring that biases the guard towards the extended position.

In another embodiment of the present invention, a sampling device configured to collect a sample from a medical device and to transfer the sample to a sample processor for processing of the sample. The sampling device includes an outer shell having an interior and a first open end. The first open end of the outer shell includes a connector that is configured to selectively seal the first open end to the sample processor and to the medical device. The sampling device further includes a sample collector at least partially disposed within the interior of the outer shell that is configured to collect the sample from the medical device. The connector is configured to removably connect the sample collector to the medical device to collect the sample and to removably connect the sample collector to the sample processor to transfer the sample to the sample processor. The sampling device further comprises an interposed port including a first opening configured to be removably connected to an access port of the medical device, and a second opening configured to be removably connected to the connector of the outer shell thereby defining a sealed connection between the outer shell and the medical device. The interposed port bifurcates an extension line of the medical device to permit flow of a fluid from the extension line to an interior of the interposed port. A first end of the sample collector is disposed within the second opening of the interposed port such that the first end is exposed to the fluid from the extension line without blocking a flow of the fluid from the extension line. The sampling device further comprising a guard movably connected to the outer shell, the guard being movable along a longitudinal axis of the outer shell between a stowed position in which the guard is retracted inwardly away from the first open end of the outer shell and an extended position in which the guard extends outwardly beyond the first open end of the outer shell to protect the sample collector. The guard includes a spring that biases the guard towards the extended position. The sample collector includes a surface that is configured to bind pathogens thereto. An interior of the medical device is comprised of a material, and the surface of the sample collector is comprised of the material that comprises the interior of the medical device. The connector is configured to removably connect the sample collector to the medical device such that the surface of the sample collector is tangential to adjacent interior surfaces of the medical device and such that the surface of the sample collector and the adjacent interior surfaces of the medical device together provide a continuous interior surface within the interior of medical device. The surface includes an absorbent material. The surface is configured to limit absorption of substances other than pathogens. The surface includes at least one of pores, through holes, and structured surfaces that increase an effective surface area of the surface. The connector is directly connected to an access port of the medical device. The connector comprises at least one of threads, tapers, and snaps. The medical device is at least one of a catheter, a scope, and a line.

In another embodiment of the present invention, a sampling device configured to collect a sample from a medical device and to transfer the sample to a sample processor for processing of the sample. The sampling device includes an outer shell having an interior and a first open end. The first open end of the outer shell includes a connector that is configured to selectively seal the first open end to the sample processor and to the medical device. The sampling device further includes a sample collector at least partially disposed within the interior of the outer shell that is configured to collect the sample from the medical device. The connector is configured to removably connect the sample collector to the medical device to collect the sample and to removably connect the sample collector to the sample processor to transfer the sample to the sample processor. The sample collector further comprises an opening provided at a first end of the sample collector and configured to communicate with and collect the sample from an interior of the medical device and further configured to communicate with and deposit the collected sample into the sample processor, an internal sample chamber configured to receive the sample, and a depressor that is configured to change an internal volume of the internal sample chamber to selectively draw the sample into and expel the sample from the internal sample chamber. The internal sample chamber is comprised of hollow interiors of the first end and the depressor of the sample collector and at least a portion of the interior of the outer shell. The depressor comprises a flexible material biased in a convex shape. The depressor is biased in an initial position via a spring that maximizes a range of depression of the depressor. The opening is provided at the first open end of the outer shell and the depressor is provided at a second end of the sample collector that opposes the opening along a longitudinal axis of the outer shell. The outer shell is an elongate tube. The sampling device further comprising a guard movably connected to the outer shell, the guard being movable along a longitudinal axis of the outer shell between a stowed position in which the guard is retracted inwardly away from the first open end of the outer shell and an extended position in which the guard extends outwardly beyond the first open end of the outer shell to protect the sample collector. The guard includes a spring that biases the guard towards the extended position. The connector is directly connected to an access port of the medical device. The connector comprises at least one of threads, tapers, and snaps. The medical device is at least one of a catheter, a scope, and a line.

In another embodiment of the present invention, the sample collector includes a first end having an opening configured to communicate with and collect the sample from an interior of the medical device. The sampling device also includes a second end that opposes the first end; the second end having an opening configured to communicate with and transfer the sample to the sample processor. The sampling device also includes an internal sample chamber configured to receive the sample. The sampling device further includes a depressor that is configured to change an internal volume of the internal sample chamber to selectively draw the sample into and expel the sample from the internal sample chamber. The internal sample chamber is included in hollow interiors of the first end, the second end, a central portion arranged between the first end and the second end, and the depressor of the sample collector. The opening of the first end of the sample collector is configured to be removably connected to the medical device to collect the sample and the opening of the second end of the sample collector is configured to be removably connected to the sample processor to transfer the sample to the sample processor. The depressor comprises a flexible material biased in a convex shape. The depressor is biased in an initial position via a spring that maximizes a range of depression of the depressor. The first end of the sample collector includes a connector that is configured to removably connect the sample collector to the medical device to collect the sample. The second end of the sample collector includes a connector that is configured to removably connect the sample collector to the sample processor to transfer the sample from the sample collector to the sample processor. The opening of the first end of the sample collector and the opening of the second end of the sample collector each include a one way valve oriented to limit flow of the sample in one direction along a path from the opening of the first end of the sample collector, through the internal sample chamber, and out the opening of the second end of the sample collector. The depressor is provided at the central portion of the sample collector, and a depression of the depressor is configured to pump the sample through the path. The sampling device further comprising an outer shell having an interior, and the sample collector is provided within the interior of outer shell. The outer shell includes a first window provided through the outer shell having a perimeter that is configured to accommodate the depressor therein, the first window being aligned with the depressor such that the depressor is configured to be depressed without interference from the outer shell. The sample collector is configured to rotate within and relative to the outer shell. The outer shell further comprises a first open end having a connector that is configured to be removably connected to the medical device to allow fluid communication between the first open end and the medical device, and a second open end having a connector that is configured to be removably connected to the sample processor to allow fluid communication between the second open end and the sample processor. In a first position, the opening of the first end of the sample collector is coaxially aligned and in fluid communication with the first open end of the outer shell and the opening of the second end of the sample collector is coaxially aligned and in fluid communication with the second open end of the outer shell. The outer shell further comprises a third open end having a connector that is configured to be removably connected to the medical device to allow fluid communication between the third open end and the medical device, and a fourth open end having a connector that is configured to be removably connected to a waste container to allow fluid communication between the fourth open end and the waste container. In a second position, the opening of the first end of the sample collector is coaxially aligned and in fluid communication with the third open end of the outer shell and the opening of the second end of the sample collector is coaxially aligned and in fluid communication with the fourth open end of the outer shell. The sample collector is configured to rotate between the first position and the second position. The sample collector includes a handle. The outer shell includes a second window provided through a portion of a perimeter of the outer shell. The handle extends through the second window and is configured to move freely within the second window upon rotation of the sample collector between the first position and the second position. The connector is directly connected to an access port of the medical device. The connector comprises at least one of threads, tapers, and snaps. The medical device is at least one of a catheter, a scope, and a line.

Another aspect of the various embodiments of the invention is directed to a sampling device configured to collect a sample and transfer the sample to a sample processor for processing of the sample. The sampling device includes an outer shell having an interior and a first open end. The first open end includes a connector that is configured to selectively provide a seal between the first open end and the sample processor. The sampling device also includes a sample collector at least partially disposed within the interior of the outer shell that is configured to move along a longitudinal axis of the outer shell. The sample collector further includes a first end configured to extend a predetermined distance beyond the first open end to collect the sample. At least a portion of the first end is configured to retract within the interior of the outer shell after sample collection. The sampling device further comprising a guard movably connected to the outer shell. The guard is movable along the longitudinal axis of the outer shell between a stowed position, in which the guard is retracted inwardly away from the first open end of the outer shell, and an extended position, in which the guard extends outwardly beyond the first open end of the outer shell to protect the sample collector. The guard includes a spring that biases the guard towards the extended position. The first end of the sample collector includes a surface that is configured to bind pathogens thereto. The surface includes an absorbent material. The surface is further configured to limit absorption of substances other than pathogens. The surface includes at least one of pores, through holes, and structured surfaces that increase an effective surface area of the surface. The connector comprises at least one of threads, tapers, and snaps. The first end of the sample collector has an outer diameter and the first open end of the outer shell has an inner diameter. The inner diameter of the first open end of the outer shell is at least twice as large as the outer diameter of the first end of the sample collector. The sample collector further includes a second end that is arranged opposite the first end in the longitudinal axis of the outer shell and a central portion arranged between the first end and the second end. The outer shell further comprises a second open end and the central portion is slideably arranged within the second open end of the outer shell. The second end of the sample collector is a handle for controlling movement of the first end of the sample collector along the longitudinal axis of the outer shell. The second end of the sample collector is configured to abut the second open end of the outer shell to define a maximum extension length of the first end of the sample collector. The central portion of the sample collector includes a seal that prevents complete removal of the sample collector from the second open end of the outer shell. The sample collector further comprises a depressor arranged at the second end and an internal sample chamber configured to receive the sample. The internal sample chamber is comprised of hollow interiors of the first end, the central portion, the second end, and the depressor of the sample collector. The first end of the sample collector includes an opening and the sample is configured to be selectively drawn into and expelled from the opening. The depressor is configured to change an internal volume of the internal sample chamber to selectively draw the sample into and expel the sample from the internal sample chamber through the opening of the first end. The depressor comprises a flexible material biased in a convex shape. The first end of the sample collector has an outer diameter and the first open end of the outer shell has an inner diameter. The inner diameter of the first open end of the outer shell is at least twice as large as the outer diameter of the first end of the sample collector.

Another aspect of the various embodiments of the invention is directed to an infection detection system comprising a sampling device. The sampling device comprises a housing having a bottom surface, a cavity, an opening extending through the bottom surface to the cavity, and an outlet. The sampling device comprises a lancet slidably disposed within the cavity and configured to be subcutaneously injected into a patient. The sampling device comprises a reservoir removably attached to the outlet of the housing, the reservoir including an opening that is configured to receive a whole blood sample and a seal that is configured to automatically seal the opening upon a detachment of the reservoir. The sampling device comprises a microfluidic passage extending from the opening of the housing to the outlet of the housing, the microfluidic passage being configured to passively draw the whole blood sample from the opening, out the outlet, and into the reservoir. The infection detection system comprises a sample processor that is configured to be attached to the reservoir and to receive and process the whole blood sample. The infection detection system comprises an analytical instrument configured to receive the sample processor and to analyze results of the processing of the whole blood sample. The internal surfaces of the sampling device include an anticoagulant coating. The internal surfaces of the sampling device include at least one of internal surfaces of the microfluidic passage and internal surfaces of the reservoir. The microfluidic passage is configured to passively draw the whole blood sample as a result of at least one of capillary and gravitational forces acting on the whole blood sample. The microfluidic passage is configured to meter a predetermined quantity of whole blood sample provided to the reservoir. The sample processor comprises a first chamber that contains lysate. The reservoir is configured to be in fluid communication with the first chamber. The sample processor comprises a second chamber that contains a first diluent and a third chamber that contains a second diluent. The sample processor comprises at least one reaction tube containing a reagent.

A further aspect of the various embodiments of the invention is directed to a method of detecting an infection in a patient treated with an external communicating medical device. The method utilizes an infection detection system including a sampling device, a sample processor, and an analytical instrument. The sampling device includes one of any of the above-described embodiments. The method includes exposing a sample collector of the sampling device to an internal environment that exists within the medical device and collecting a sample from the internal environment over a period of time during which the patient is treated with the medical device. The method further includes removing the sampling device from the medical device. The method also includes connecting the sampling device to the sample processor and processing the sample collected by the sample collector via the sample processor. The method also includes connecting the sample processor to the analytical instrument and analyzing the processed sample via the analytical instrument. The method includes exposing, subsequent to the removal of the sampling device from the medical device, another sampling device to the internal environment and collecting another sample from the internal environment over a second period of time during which the patient is treated with the medical device. The exposing of the sample collector to the internal environment includes arranging a surface of the sample collector at a position tangential to adjacent interior surfaces of the medical device such that the surface of the sample collector and the adjacent interior surfaces of the medical device together provide a continuous interior surface within an interior of medical device. The exposing of the sample collector to the internal environment includes providing the surface of the sample collector with a material that is the same as a material that comprises the adjacent interior surfaces of the medical device. The method includes detecting that the patient displays a symptom of the infection prior to the removing of the sampling device from the medical device. Connecting of the sampling device to the sample processor occurs directly after removing the sampling device from the medical device. The subcutaneously collecting the whole blood sample includes passively drawing the whole blood sample through a microfluidic passage of the sampling device. The method comprising automatically exposing the whole blood sample to an anticoagulant that is coated onto an interior surface of the sampling device. The interior surface of the sampling device includes at least one of an interior surface of the reservoir and an interior surface of a microfluidic passage of the sampling device. The processing the whole blood sample in the sample processor comprises mixing lysate, a first diluent, and the whole blood sample in a first chamber. The processing the whole blood sample in the sample processor comprises hydrating reagents contained within at least one reaction tube of the sample processor with a second diluent. The mixing of the lysate, the first diluent, and the whole blood sample in the first chamber occurs concurrently with the hydrating of the reagents contained within the at least one reaction tube. The mixing of the lysate, the first diluent, and the whole blood sample in the first chamber and the hydrating of the reagents contained within the at least one reaction tube occurs for a predetermined period of time. The processing the whole blood sample in the sample processor further comprises supplying the lysate, the first diluent, and the whole blood sample to the at least one reaction tube upon expiration of the predetermined period of time. The collecting of the whole blood sample in the reservoir includes metering the whole blood sample to collect a predetermined quantity of the whole blood sample.

Further aspects of the various embodiments of the invention are directed to a method of detecting an infection in a patient using an infection detection system comprising a sampling device, a sample processor, and an analytical instrument. The method comprises subcutaneously collecting a whole blood sample in a reservoir of the sampling device. The method comprises connecting the reservoir to the sample processor and transferring the whole blood sample from the reservoir to the sample processor. The method comprising processing the whole blood sample in the sample processor. The method comprising connecting the sample processor to the analytical instrument and analyzing the processed sample via the analytical instrument. The method comprises performing the entire method in proximity to the patient. Proximity to the patient includes in a single facility. Proximity to the patient includes in a single room. The subcutaneously collecting the whole blood sample includes injecting the patient with a lancet of the sampling device. The method comprising removing the reservoir and concurrently sealing the reservoir. The connecting the reservoir to the sample processor includes automatically terminating the sealing of the reservoir. The sealing the reservoir occurs automatically via a seal that seals an opening of the reservoir as a consequence of the removing the reservoir, and the removing the reservoir includes removing the reservoir from an outlet of the sampling device. The method comprising maintaining the sealing of the reservoir subsequent the removing of the reservoir and terminating the sealing of the reservoir upon the connecting of the reservoir to the sample processor.

There are, of course, additional aspects of the various embodiments of the invention disclosed herein that will be described below and which will form the subject matter of the claims. In this respect, before explaining at least one aspect of the invention in detail, it is to be understood that the invention is not limited in its application to the details of construction and to the arrangements of the components set forth in the following description or illustrated in the drawings. The invention is capable of aspects in addition to those described and of being practiced and carried out in various ways. Also, it is to be understood that the phraseology and terminology employed herein, as well as the Abstract, are for the purpose of description and should not be regarded as limiting.

As such, those skilled in the art will appreciate that the conception upon which this invention is based may readily be utilized as a basis for the designing of other structures, methods, and systems for carrying out the several purposes of the invention. It is important, therefore, that the claims be regarded as including such equivalent constructions insofar as they do not depart from the spirit and scope of the invention.

### BRIEF DESCRIPTION OF DRAWINGS

In order that the invention may be readily understood, aspects of the invention are illustrated by way of examples in the accompanying drawings; however, the subject matter is not limited to the disclosed aspects.

FIGS. 1A-1E illustrate various stages of use of an exemplary infection detection system in accordance with aspects of the invention.

FIGS. 2A-2C illustrate a first exemplary embodiment of a sampling device for use with the infection detection system in accordance with aspects of the invention.

FIG. 3 illustrates an exemplary process for detecting an infection that utilizes an infection detection system in accordance with aspects of the invention.

FIGS. 4A-4C illustrate an exemplary interposed port for use with an infection detection system in accordance with aspects of the invention.

FIGS. 5A-7B illustrate a second exemplary embodiment of the sampling device for use with the infection detection system in accordance with aspects of the invention.

FIGS. 8-10C illustrate an exemplary tool for use with the infection detection system in accordance with aspects of the invention.

FIGS. 11-12C illustrate a third exemplary embodiment of the sampling device for use with the infection detection system in accordance with aspects of the invention.

FIGS. 13A and 13B illustrate a fourth exemplary embodiment of the sampling device for use with the infection detection system in accordance with aspects of the invention.

FIGS. 14A and 14B illustrate a fifth exemplary embodiment of the sampling device for use with the infection detection system in accordance with aspects of the invention.

FIGS. 15A-16C illustrate a sixth exemplary embodiment of the sampling device for use with the infection detection system in accordance with aspects of the invention.

FIGS. 17A and 17B illustrate a seventh exemplary embodiment of the sampling device for use with the infection detection system in accordance with aspects of the invention.

FIG. 18 illustrates an eighth exemplary embodiment of the sampling device for use with the infection detection system in accordance with aspects of the invention.

FIG. 19 illustrates an exemplary embodiment of the sample processor for use with the infection detection system in accordance with aspects of the invention.

FIG. 20 illustrates an exemplary process for detecting an infection that utilizes the infection detection system in accordance with aspects of the invention.

Features of the infection detection system according to aspects of the invention are described with reference to the drawings, in which like reference numerals refer to like parts throughout.

### DETAILED DESCRIPTION

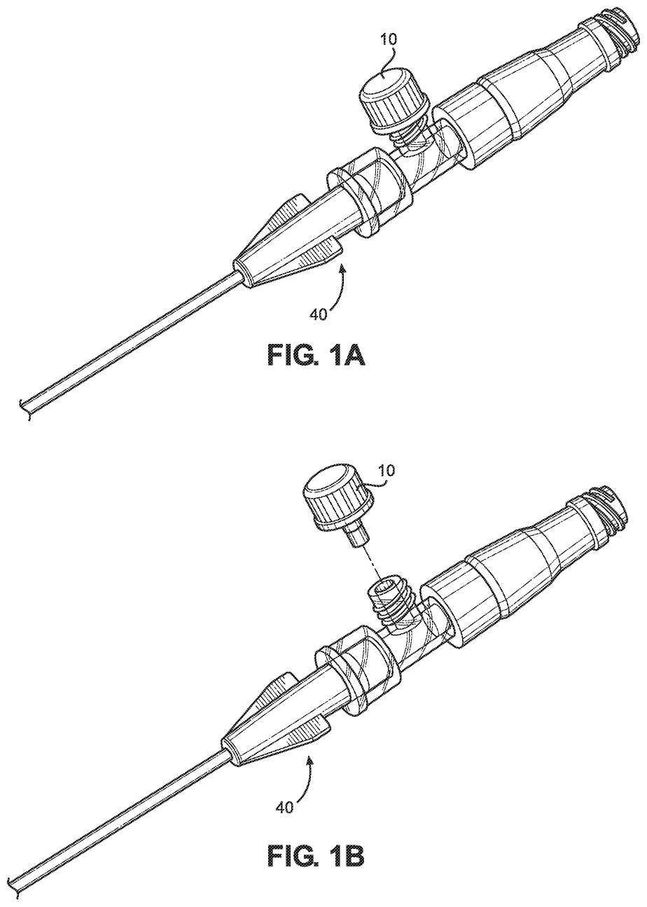

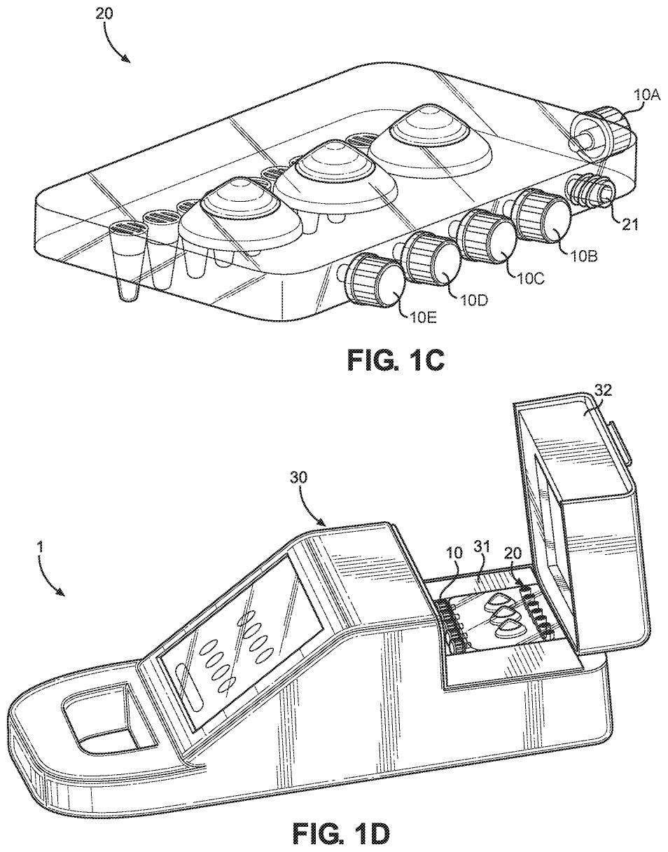

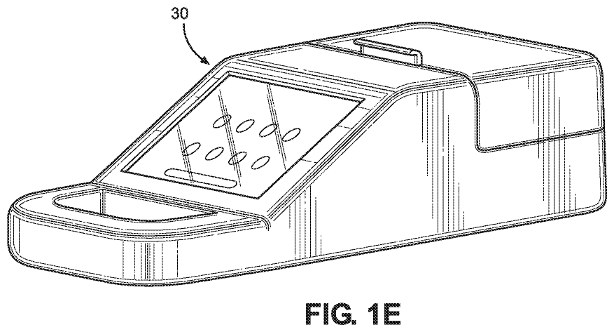

FIGS. 1A-1E illustrate various stages of use of an exemplary infection detection system 1 of the invention; the infection detection system 1 comprising a sampling device 10, a sample processor 20, and an analytical instrument 30. FIG. 1A shows the sampling device 10 connected to a medical device 40. FIG. 1B shows the sampling device 10 removed from the medical device 40. FIG. 1C shows a plurality of exemplary sampling devices 10a-10d connected to the sample processor 20. FIG. 1D shows the sampling device 10 connected to the sample processor 20 received within the analytical instrument 30. FIG. 1E shows the analytical instrument 30 analyzing a processed sample.

As shown in FIGS. 1A-1E, the sampling device 10 of the infection detection system 1 may be adapted to collect a sample, such as blood (e.g., whole blood), urine, fecal matter, purulence/pus, etc. The sampling device 10 may collect the sample from a medical device 40. For example, the sampling device 10 may be exposed for a predetermined and/or extended period of time to an internal space or lumen in the medical device 40 so as to collect a sample of any pathogen which may form in said space and/or lumen. The medical device 40 may be an external communicating device used for treating a patient, such as a Foley catheter, a vascular catheter, a suction catheter, a bronchial scope, a urinary drain line, a respiratory suction catheter, a Bronco-Alveolar-Lavage Catheter, etc. The sampling device 10 may additionally or alternatively be adapted to collect a sample directly from a sample source such as urine, fecal matter, purulence/pus, a suspected infection site (such as a surgical dressing, wound, and/or an insertion site), etc. The sampling device 10 may be disposable and replaceable. Details of exemplary embodiments of the sampling devices of the infection detection system 1 in accordance with the invention are provided below.

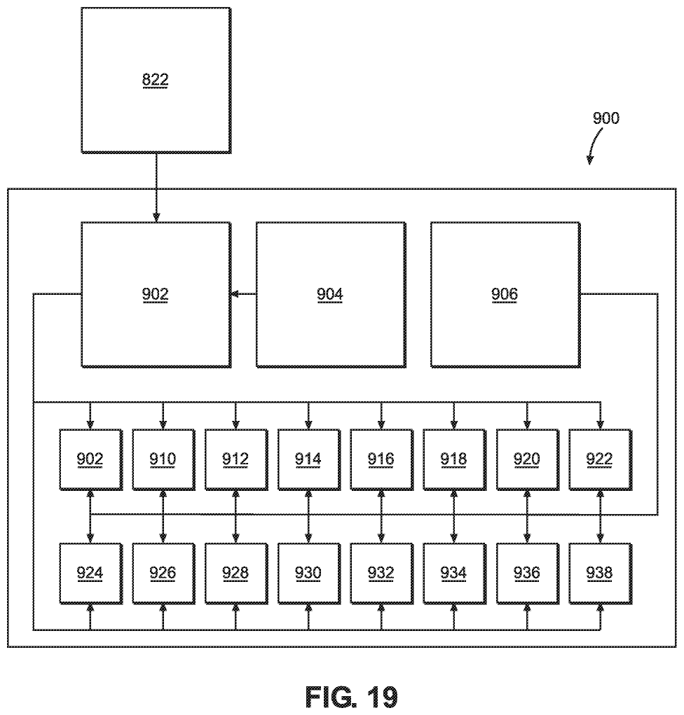

The sample processor 20 of the infection detection system 1 may be removably connected to the sampling device 10 and may receive the collected sample from the sampling device 10. The sample processor 20 may be disposable and replaceable, and may be adapted to process the collected sample. In general, the sample processor 20 may be configured to include some or all of the reagents necessary to perform any suitable nucleic acid sequence-based amplification (NASBA)-based nucleic-acid assay for mRNA and/or DNA on the sample. For example, the sample processor 20 may include all of the reagents/structures/materials for lysing pathogen cells and extracting and purifying pathogen messenger RNA (i.e., dissolve targeted mRNA and remove inhibitors that could interfere with nucleic acid amplification). In another example, the sample processor 20 may include all of the reagents/structures/materials for processing the output solution from the extraction and purification steps for isothermal amplification of targeted mRNA to identify the presence of specific genes. Specific examples of reagents include lysing buffers, mRNA-dependent DNA polymerase, mRNA primers, DNA primers, amino acids, and the like. Specific examples of structures includes reagent supply chambers, conduits for fluid transfer, reaction chambers, and the like. Specific examples of materials includes replacement sampling devices 10a-10e, thermally conductive surfaces for reaction chambers, and the like. An illustrative exemplary embodiment of a sample processor 900 of the infection detection system 1 in accordance with the invention is illustrated in FIG. 19 and described below.

The analytical instrument 30 of the infection detection system 1 may be adapted to receive the sample processor 20 and to analyze the processed sample. The analytical instrument 30 may be configured to perform any suitable NASBA-based nucleic-acid assay on the sample utilizing the reagents. For example, the analytical instrument 30 may be configured to perform any steps for lysing pathogen cells and extracting and purifying pathogen messenger RNA (i.e., dissolve targeted mRNA and remove inhibitors that could interfere with nucleic acid amplification). In another example, the analytical instrument 30 may be configured to perform any steps for processing the output solution from the extraction and purification steps for isothermal amplification of targeted mRNA to identify the presence of specific genes.

In a particular example, the sample processor 20 may be configured to automatically process the sample in response to the sample being introduced to, e.g., an access port 21 of the sample processor 20 and/or in response to the sample processor 20 being introduced to a receptacle 31 of the analytical instrument 30. In this regard, closing a lid 32 of the analytical instrument 30 may urge the reagents past the sampling device 10 with collected sample. The sample may be automatically directed via conduits (not shown) to a reaction chamber (not shown) of the sample processor 20 and the analytical instrument 30 may automatically trigger the processing of the collected sample in the sample processor 20 and analyze the processed sample. An illustrative exemplary embodiment of sample processing in accordance with aspects of the invention is described below.

According to aspects of the invention, the entirety of the sample processing may occur within the various components of the infection detection system 1 thereby obviating the need of direct user interaction with the sample to effectuate sample processing. The infection detection system 1 may accordingly be used by a user of low skill and may be readily transported to and applied in a variety of environments (e.g., the home, a hospital room, etc.). As a result, infection in a patient may be rapidly detected and identified, which may improve the prognosis of the patient.

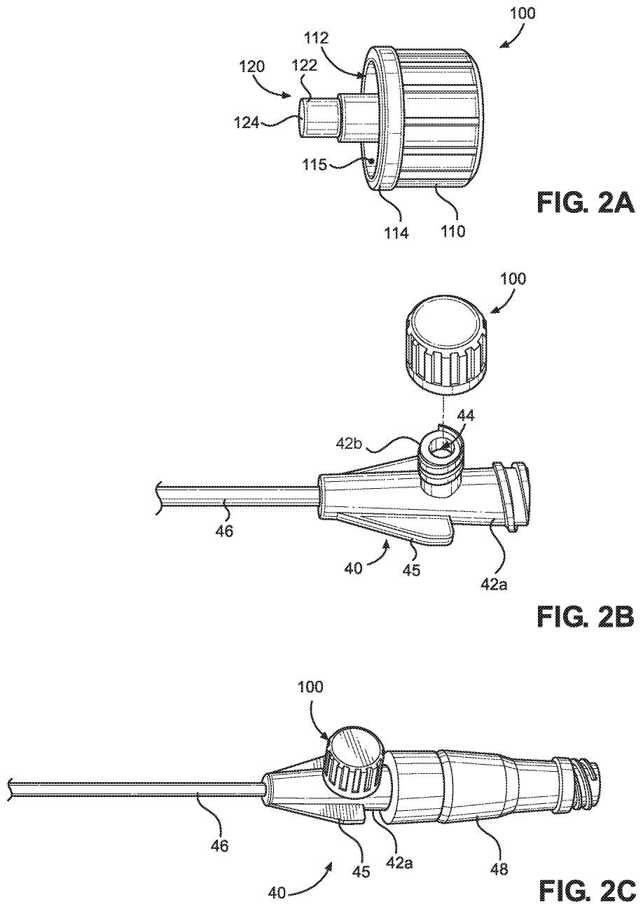

FIGS. 2A-2C illustrate a first exemplary embodiment of the sampling device 100, which is a removable witness to the medical device 40. FIG. 2A shows a perspective view of the sampling device 100. FIG. 2B shows the sampling device 100 disconnected from the medical device 40. FIG. 2C shows the sampling device 100 connected to the medical device 40.

As illustrated in FIG. 2A, the sampling device 100 may include an outer shell 110 having an interior 112 and a first open end 114. The outer shell 110 may include a geometry that facilitates grasping of the outer shell 110 during attachment/removal of the sampling device 100, such as via a pulling, a squeezing, and/or a threading of the sampling device 100. The first open end 114 of the outer shell 110 may include a connector 115 (depicted schematically in FIG. 2A), which may be provided on an interior of the first open end 114, that may selectively seal the first open end 114 to the sample processor (not shown) and to the medical device (not shown). The connector 115 may removably connect a sample collector 120 (described below) of the sampling device 100 to the medical device to collect the sample and may removably connect the sample collector 120 to the sample processor to transfer the collected sample to the sample processor. For example, the connector 115 may include at least one of male/female luer fittings, threads, tapers, snaps, splines, and O-rings adapted to connect to complimentary structures of the medical device and the sample processor. Accordingly, the connector 115 may be selectively snap-fit, press-fit, and/or threaded to the complimentary structures of the medical device and the sample processor to realize the removable connections thereto. The removable connections between the connector 115 and the medical device/sample processor may form a seal. According to aspects of the invention, the sampling device 100 may be removably connected to and provide a seal with the medical device without disrupting the function of the medical device (i.e., without reducing flow, without forming air pockets, and/or without generating interrupted surfaces within the medical devices), which reduces the risk of thrombus formation in the patient treated by the medical device and/or the development of unintended bacterial growth.

The sample collector 120 of the sampling device 100 may be at least partially disposed within the interior 112 of the outer shell 110 and may collect the sample from the medical device. The sample collector 120 may include an end 122 that is a removable witness to an internal environment existing within the medical device. The end 122 of the sample collector 120 may include a surface 124 adapted to specifically or non-specifically bind pathogens thereto. In embodiments, the surface 124 may be comprised of the same material as a material that comprises at least a portion of the interior of the medical device such that the sample collector 120 may serve as a removable witness to the internal environment existing within the medical device. Alternatively, the surface 124 may be comprised of an immobilization of an antibody (e.g., anti-Staphylococcus aureus, anti-Salmonella, and/or anti Shigella antibody), a segment of antibody that attracts pathogens, and/or a moiety that binds to a surface receptor of a pathogen (e.g., mannose binding lectin, RNAs, DNAs, and ss-DNAs). The surface 124 may additionally or alternatively be comprised of an absorbent material (e.g., a swab or sponge). The surface 124 may additionally or alternatively be adapted to limit absorption of substances other than pathogens. For example, the surface 124 may include a non-fouling background (e.g., a non-fouling polymer with functional groups for specific modification) that reduces nonspecific protein binding, thrombus formation, and/or mammalian cell attachment. The surface 124 may additionally or alternatively include at least one of pores, through holes, and structured surfaces (e.g., on the micro or nano level) that increase an effective surface area of the surface 124 and to which the sample may adhere/absorb.

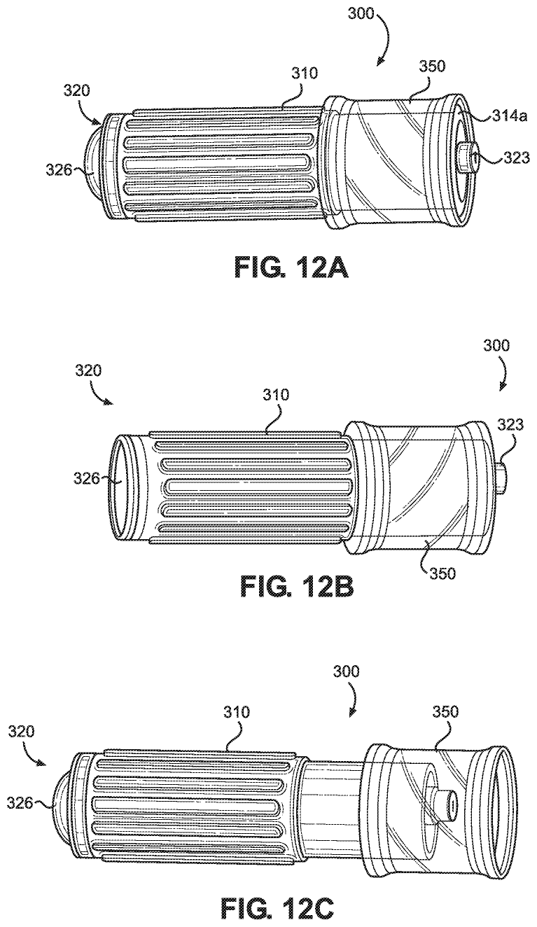

In embodiments (not shown), the sampling device 100 may include a guard movably connected to the outer shell 110. The guard may be movable along a longitudinal axis of the outer shell 110 between a stowed position in which the guard is retracted inwardly away from the first open end 114 of the outer shell 110 and an extended position in which the guard extends outwardly beyond the first open end 114 of the outer shell 110 to protect the sample collector 120. The guard may include a spring that biases the guard towards the extended position. The guard of the sampling device 100 may, for example, be similar to the guard 350 of the sampling device 300 shown in FIGS. 11-12C and described in detail below.

As shown in FIGS. 2B and 2C, the sampling device 100 may be directly connected to at least one of first and second access ports 42a, 42b of a luer hub 45 of the medical device 40 to expose the sample collector 120 to an interior 44 (e.g., space or lumen) of the medical device 40. The first access port 42a may be connected to an accessory such as a cap 48 (e.g., a needleless connector). Alternatively, the sampling device 100 may be connected to any exposed portion of the medical device 40 that is external to the patient. The luer hub 45 may be connected to an extension line 46, such as a luer. As shown in FIG. 2C, the sampling device 100 may be directly connected to the second access port 42b of the luer hub 45 that bifurcates and that may be perpendicular to the extension line 46. The connector 115 may be connected to the medical device 40 and may hold the surface 124 of the sample collector 120 in a position tangential to adjacent interior surfaces (not shown) of the medical device 40 such that the surface 124 of the sample collector 120 and the adjacent interior surfaces of the medical device 40 together provide a continuous interior surface (not shown) within the medical device 40. That is, the surface 124 may be adapted so as to not interrupt an internal geometry of the medical device 40 when the sampling device 100 is connected to the medical device 40. According to aspects of the invention, the continuous interior surface provided by the surface 124 of the sample collector 120 and the adjacent interior surfaces of the medical device 40 prevents the formation of interrupted surfaces within the interior of the luer hub 45, which may form a habitus for unintended bacterial growth.

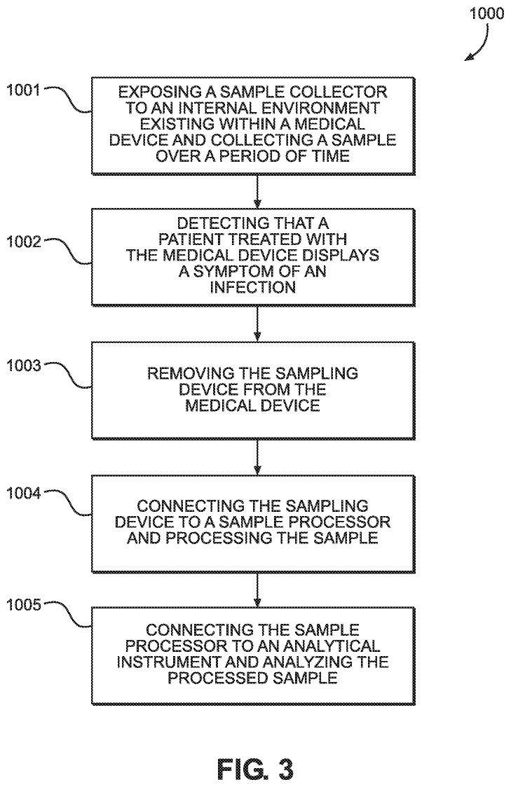

FIG. 3 illustrates an exemplary infection detection process 1000 that utilizes an infection detection system including the first exemplary embodiment of the sampling device 100, described above and illustrated in FIGS. 2A-2C. The infection detection process 1000 may be used for detecting an infection in a patient that is treated with an external communicating medical device, examples of which are provided above. In addition to the sampling device 100, the infection detection system utilized by the infection detection process 1000 may further include the sample processor 20 and the analytical instrument 30, as described above.

As shown in FIG. 3, at step 1001 of the infection detection process 1000 a user may expose the sample collector 120 of the sampling device 100 to an internal environment that exists within the medical device. The sample collector 120 may collect a sample from the internal environment over a period of time during which the patient is treated with the medical device. For example, the sample collector 120 may act as a removable witness to the internal environment within the medical device over a period of time extending from the moment the patient starts treatment with the medical device to the moment the sample collector 120 is removed for processing (as discussed below). The period of time may alternatively be a prolonged period of time sufficient for the sample collector 120 to capture a representative sample of the internal environment existing, for example, on internal surfaces of the medical device. The exposing of the sample collector 120 to the internal environment may further include arranging a surface 124 of the sample collector 120 at a position tangential to adjacent interior surfaces of the medical device such that the surface 124 of the sample collector 120 and the adjacent interior surfaces of the medical device together provide a continuous interior surface within the interior of the medical device. The exposing of the sample collector 120 to the internal environment may further include providing the surface 124 of the sample collector 120 with a material that is the same as a material that comprises the adjacent interior surfaces of the medical device such that the sampling device 100 may act as a removable witness to the internal environment.

At step 1002, a user and/or device may manually and/or automatically (in the case of a device) detect that the patient displays a symptom of the infection. For example, the patient may exhibit an elevated body temperature, an elevated heart rate, etc. At step 1003, the user may remove, subsequent to the detection of the symptom of infection, the sampling device 100 from the medical device. The infection detection process 1000 may further include a user exposing, subsequent to the removal of the sampling device 100 from the medical device, another sampling device 100 in accordance with the first exemplary embodiment, to the internal environment and collecting another sample from the internal environment over a second period of time during which the patient is treated with the medical device. The second period of time may also be a prolonged period of time sufficient for the sample collector 120 to capture a representative sample of the internal environment existing, for example, on the internal surfaces of the medical device.

At step 1004, the sampling device 100 may be connected to the sample processor 20 and the sample processor 20 may process the sample collected by the sample collector 120, as discussed above. The sampling device 100 may be directly connected to the sample processor 20 immediately upon or a short period of time after removal from the medical device. That is, the sampling device 100 may be directly connected to the sample processor 20 after removal from the medical device without any intervening steps (e.g., treatment, storage, and/or extraneous transport) within a period of time for a typical user to traverse the distance between the medical device and the sample processor 20 to connect the sampling device 100 thereto. By directly connecting the sampling device 100 to the sample processor 20, processing of the sample may be rapidly performed (e.g., in less than 30 minutes) at the location that the sample is collected, eliminating the need for time-wasting intermediary treatment, storage, and/or extraneous transport of the sampling device 100. At step 1005, the sample processor 20 may be connected to the analytical instrument 30 and the analytical instrument 30 may analyze the processed sample, as discussed above. The sample processor 20 may be directly connected to the analytical instrument 30 immediately or a short period of time after the sampling device 100 is connected to the sample processor 20. That is, the sample processor 20 may be directly connected to the analytical instrument 30 after the sampling device 100 is connected to the sample processor 20 without any intervening steps (e.g., treatment, storage, and/or extraneous transport) within a period of time for a typical user to traverse the distance between the sample processor 20 and the analytical instrument 30 to connect the sample processor 20 thereto. By directly connecting the sample processor 20 to the analytical instrument 30, analysis of the sample may be rapidly performed (e.g., in less than 30 minutes) at the location that the sample is collected, eliminating the need for time-wasting intermediary treatment, storage, and/or extraneous transport of the sample processor 20.

Though features of the infection detection process 1000 are specifically adapted for use with the first exemplary embodiment of the sampling device 100, as would be readily apparent to those skilled in the art, similar infection detection processes may be specifically adapted for use with the unique and/or common features of each of the exemplary embodiments of the sampling devices described herein.

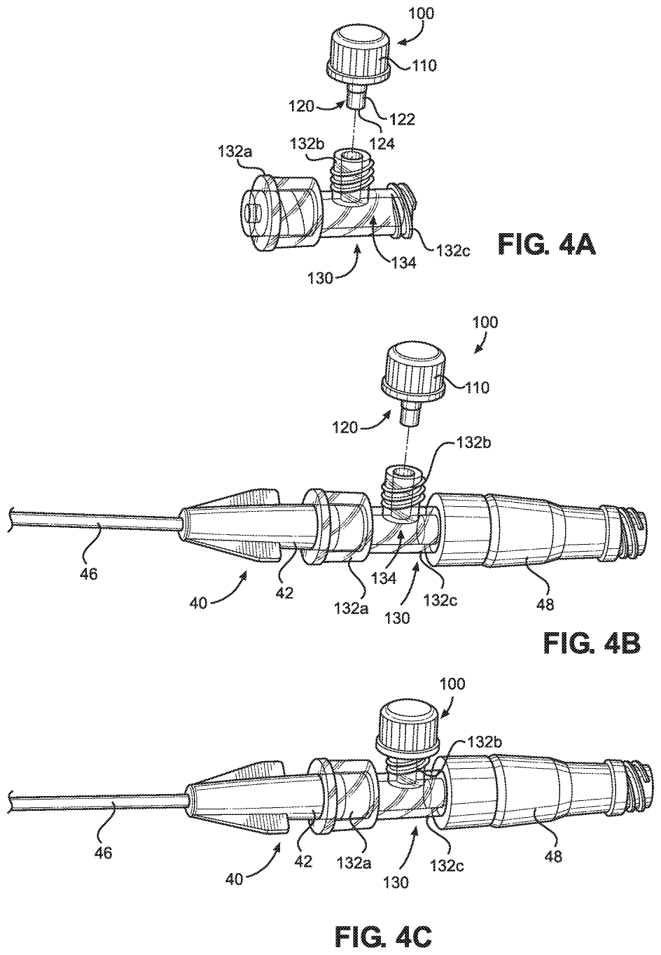

FIGS. 4A-4C illustrate an exemplary interposed port 130, for use with the infection detection system 1, that is adapted to connect to the medical device 40 to provide access to an internal environment existing within the medical device 40. FIG. 4A shows the sampling device 100 disconnected from the interposed port 130. FIG. 4B shows the interposed port 130 connected to the medical device 40 and provides another view of the sampling device 100 disconnected from the interposed port 130. FIG. 4C shows the interposed port 130 connected to the medical device 40 and to the sampling device 100.

As shown in FIG. 4A, the interposed port 130 may include a first opening 132a that may be removably connected to an access port of the medical device (not shown) to permit fluid communication between the internal environment existing within the medical device and an interior 134 of the interposed port 130.

The interposed port 130 may further include a second opening 132b and a third opening 132c. The second opening 132b and/or the third opening 132c may be removably connected to the connector (not shown) of the outer shell 110 of the sampling device 100 to provide a seal between the outer shell 110 and the interposed port 130. By sealing the interposed port 130 to the sampling device 100, the interposed port 130 may expose the surface 124 of the end 122 of the sample collector 120 to the internal environment existing within the medical device that is in fluid communication with the interior 134 of the interposed port 130. The interposed port 130 may additionally or alternatively expose other accessories, such as the second, third, fourth, and/or fifth exemplary embodiments of the sampling devices described in detail below, to the internal environment existing within the medical device. In addition, the first, second and third openings 132a, 132b, 132c may each include complimentary structures that facilitate connection to the medical device, the sampling devices, and/or the other accessories. The complimentary structures may be, e.g., male/female luer fittings, threads, tapers, snaps, splines, and O-rings.

As shown in FIGS. 4B and 4C, the interposed port 130 may be connected to an access port 42 of the medical device 40 and may bifurcate an extension line 46 of the medical device 40 to permit flow of a fluid from the extension line 46 to the interior 134 of the interposed port 130. The sample collector 120 may be arranged over the second opening 132b of the interposed port 130 prior to connection with the outer shell 110. The third opening 132c of the interposed port 130 may be connected to a cap 48 (e.g., a needleless connector).

As shown in FIG. 4C, the sampling device 100 may be connected to the second opening 132b of the interposed port 130 such that the first end of the sample collector (not shown) is disposed within the second opening 132b of the interposed port 130 and is exposed to the fluid from the extension line 46 in the interior 134 of the interposed port 130 without blocking the flow of the fluid from the extension line 46. That is, the surface of the first end of the sample collector may be arranged in a position tangential to adjacent interior surfaces (not shown) of the interposed port 130 such that the surface of the sample collector and the adjacent interior surfaces of the interposed port 130 together provide a continuous interior surface (not shown) within the interior 134 of the interposed port 130. According to this configuration, the surface of the sample collector may be adapted so as to not interrupt an internal geometry of the interposed port 130 when the sampling device 100 is connected to the interposed port 130. By implementing aspects of the invention, the continuous interior surface provided by the surface of the sample collector and the adjacent interior surfaces of the interposed port 130 may prevent the formation of interrupted surfaces within the interior of the interposed port 130, which may form a habitus for unintended bacterial growth.

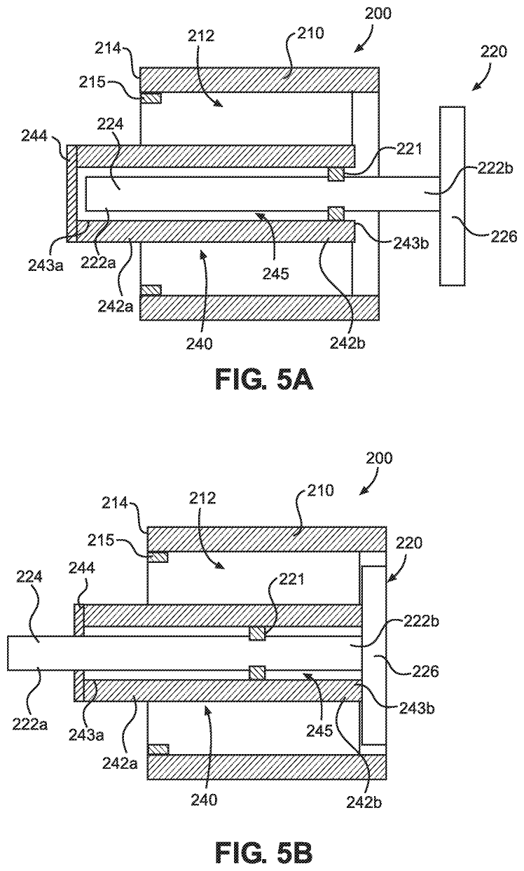

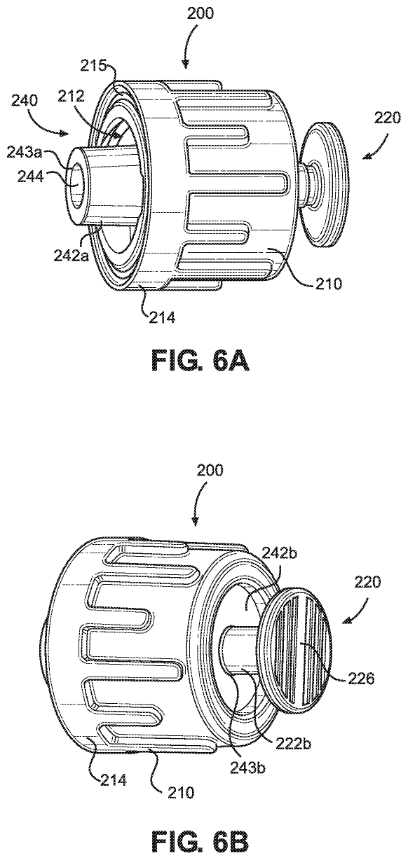

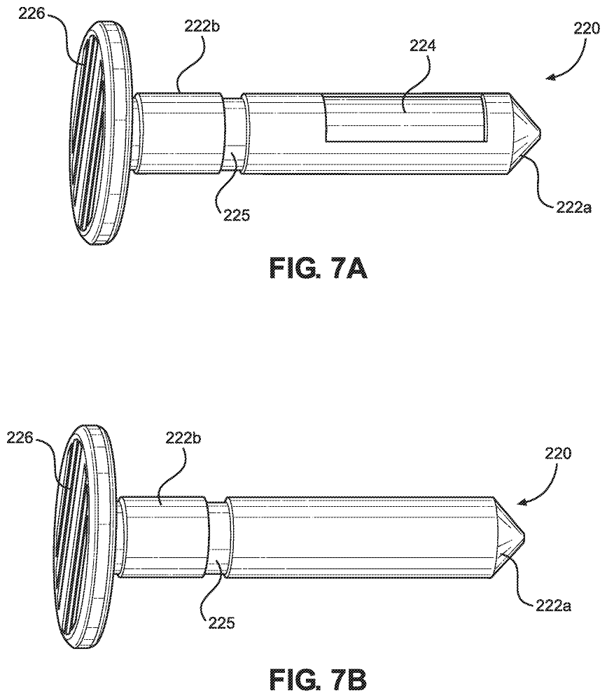

FIGS. 5A-7B illustrate a second exemplary embodiment of a sampling device 200 that serves as a selectively deployable removable witness to the medical device (not shown). The sampling device 200 according to the second embodiment enables a user to acquire a sample from the medical device (not shown) at a specified time, such as during a portion of a medical procedure. FIG. 5A shows a schematic cross-sectional view of the sampling device 200 in a retracted position. FIG. 5B shows a schematic cross-sectional view of the sampling device 200 in a deployed position. FIGS. 6A and 6B show perspective views of the sampling device 200 in the retracted position. FIGS. 7A and 7B show perspective views of a sample collector 220 of the sampling device 200.

As shown in FIGS. 5A and 5B, the sampling device 200 may include an outer shell 210 having an interior 212 and a first open end 214. The outer shell 210 may include a geometry that facilitates grasping of the outer shell 210 during attachment/removal of the sampling device 200, such as via a pulling, a squeezing, and/or a threading of the sampling device 200. The first open end 214 of the outer shell 210 may include a connector 215. The connector 215 may be provided on an interior of the first open end 214 and may selectively seal the first open end 214 to the sample processor (not shown) and to the medical device (not shown). The connector 215 may removably connect a sample collector 220 (described below) of the sampling device 200 to the medical device to collect the sample and may removably connect the sample collector 220 to the sample processor to transfer the collected sample to the sample processor. For example, the connector 215 may include at least one of male/female luer fittings, threads, tapers, snaps, splines, and O-rings adapted to connect to complimentary structures of the medical device and the sample processor. Accordingly, the connector 215 may be selectively snap-fit, press-fit, and/or threaded to the complimentary structures of the medical device and the sample processor to realize removable connections thereto. The removable connections between the connector 215 and the medical device/sample processor may form a seal. According to aspects of the invention, the sampling device 200 may be removably connected to and provide a seal with the medical device without disrupting the function of the medical device (i.e., without reducing flow, without forming air pockets, and/or without generating interrupted surfaces within the medical devices), which reduces the risk of thrombus formation in the patient treated by the medical device and/or the development of unintended bacterial growth.

The sample collector 220, shown in FIGS. 7A and 7B, of the sampling device 200 may be at least partially disposed within the interior 212 of the outer shell 210 and may collect the sample from the medical device. The sample collector 220 may include a first end 222a that may be a removable witness to an internal environment existing within the medical device. The first end 222a of the sample collector 220 may include a surface 224 that may be adapted to specifically or non-specifically bind pathogens thereto. In embodiments, the surface 224 may be comprised of the same material as a material that comprises at least a portion of the interior of the medical device such that the sample collector 220 may serve as a removable witness to an internal environment existing within the medical device. Alternatively, the surface 224 may be comprised of an immobilization of an antibody (e.g., anti-Staphylococcus aureus, anti-Salmonella, and/or anti Shigella antibody), a segment of antibody that attracts pathogens, and/or a moiety that binds to a surface receptor of a pathogen (e.g., mannose binding lectin, RNAs, DNAs, and ss-DNAs). The surface 224 may additionally or alternatively be comprised of an absorbent material (e.g., a swab or sponge). The surface 224 may additionally or alternatively be adapted to limit absorption of substances other than pathogens. For example, the surface 224 may include a non-fouling background (e.g., a non-fouling polymer with functional groups for specific modification) that reduces nonspecific protein binding, thrombus formation, and/or mammalian cell attachment. The surface 224 may additionally or alternatively include at least one of pores, through holes, and structured surfaces (e.g., on the micro or nano level) that increase an effective surface area of the surface 224 and to which the sample may adhere/absorb.

The sample collector 220 may further include a second end 222b. The second end 222b may include a depressor 226, which may for example be a handle. The second end 222b may further include a groove 225, as shown in FIGS. 7A and 7B.

As shown particularly in FIGS. 5A and 5B, at least a portion of the sample collector 220 may selectively extend and retract along a longitudinal axis of the outer shell 210 such that the surface 224 of the sample collector 220 may be selectively exposed to the internal environment existing within the medical device. To this end, the sampling device 200 may further include an isolation housing 240 that may be disposed within the interior 212 of the outer shell 210. The isolation housing 240 may include a first end 242a that may be disposed at the first open end 214 of the outer shell 210. The isolation housing 240 may further include a second end 242b disposed opposite the first end 242a with respect to the longitudinal axis of the outer shell 210. The isolation housing 240 may also include an interior 245 provided between the first and second ends 242a, 242b of the isolation housing 240 and the sample collector 220 may be at least partially provided within the interior 245 of the isolation housing 240.

The first end 242a of the isolation housing 240 may further include an opening 243a that may be closed by a protective barrier 244 (e.g., a foil) that may seal and isolate the interior 245 of the isolation housing 240. The first end 222a of the sample collector 220 may rupture the protective barrier 244 upon an extension of the sample collector 220 through the opening 243a of the first end 242a of the isolation housing 240 to selectively expose the first end 222a of the sample collector 220 to the sample, as shown in FIG. 5B. The rupture of the protective barrier 244 may be caused by a user applying a force to the depressor 226.

The second end 242b of the isolation housing 240 may include an opening 243b, and the sample collector 220 may be slideably provided within the opening 243b. The sample collector 220 may include a seal 221, as shown in FIGS. 5A and 5B, disposed between an outer surface of the sample collector 220, such as within the groove 225, and an inner surface of the isolation housing 240 to prevent the sample from passing through the opening 243b of the second end of 242b the isolation housing 240.

In embodiments (not shown), the sampling device 200 may include a guard movably connected to the outer shell 210. The guard may be movable along a longitudinal axis of the outer shell 210 between a stowed position in which the guard is retracted inwardly away from the first open end 214 of the outer shell 210 and an extended position in which the guard extends outwardly beyond the first open end 214 of the outer shell 210 to protect the sample collector 220. The guard may include a spring that biases the guard towards the extended position. The guard of the sampling device 200 may, for example, be similar to the guard 350 of the sampling device 300 shown in FIGS. 11-12C and described in detail below.

The sampling device 200 may be directly connected to at least one access port of the medical device (not shown) that may expose the sample collector 220 to an interior of the medical device. Alternatively, the sampling device 200 may be connected to the interposed port (as described above) or to any exposed portion of the medical device that is external to the patient. The connector 215 may be connected to the medical device and may hold the protective barrier 244 of the isolation housing 240 in a position tangential to adjacent interior surfaces (not shown) of the medical device such that the protective barrier 244 of the isolation housing 240 and the adjacent interior surfaces of the medical device together provide a continuous interior surface (not shown) within the medical device. That is, at least prior to rupture, the protective barrier 244 of the isolation housing 240 may be adapted so as to not interrupt an internal geometry of the medical device when the sampling device 200 is connected to the medical device. According to aspects of the invention, the continuous interior surface provided by the protective barrier 244 of the isolation housing 240 and the adjacent interior surfaces of the medical device prevents the formation of interrupted surfaces within the interior of medical device, which may form a habitus for unintended bacterial growth. The connector 215 may further be adapted such that after sample collection, the first end 222a of the sample collector 220 may be locked within the interior 245 of the isolation housing 240 until such time as the sampling device 200 is connected to the sample processor. That is, the sampling device 200 may be adapted such that, after sample collection and a subsequent retraction of the sample collector 220, only upon a connection of the sampling device 200 to the sample processor may the sample collector 220 be released from a locked position within the interior 245 of the isolation housing 240. After being released from the locked position, the first end 222a of the sample collector 220 may freely extend from the opening 243a of the first end 242a of the isolation housing 240 to deposit the sample in the sample processor for processing.

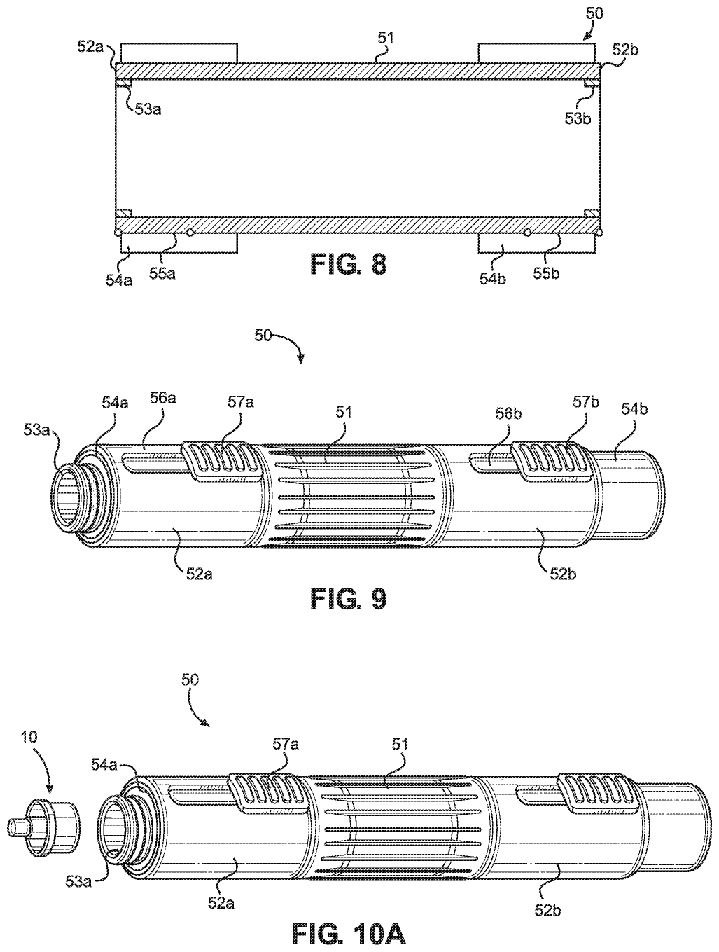

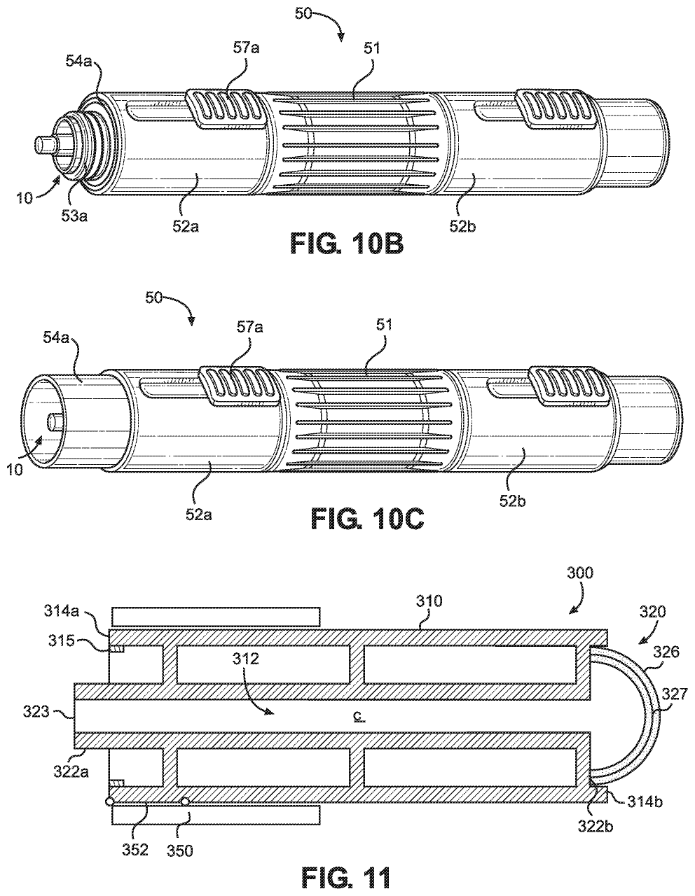

FIGS. 8-10C illustrate an exemplary tool 50 adapted to connect to and to hold the sampling device 10. The tool 50 is adapted to transfer the sampling device 10 (e.g., one or both of the sampling devices 100, 200 described above) from the medical device (not shown) to the sample processor (not shown) to promote safe, hands-free handling of the sampling device, to reduce the risk of contamination of the collected sample, and/or to prevent mishandling of small, difficult to hold sampling devices. FIG. 8 shows a schematic cross-sectional view of the tool 50. FIG. 9 shows a perspective view of the tool 50. FIG. 10A shows the sampling device 10 disconnected from the tool 50. FIG. 10B shows the sampling device 10 connected to the tool 50. FIG. 10C shows the sampling device 10 connected to and held by the tool 50 and further shows a guard 54a of the tool 50 in an extended position.

As shown in FIG. 8, the tool 50 may be a disposable structure that may include an elongate body 51. The elongate body 51 may have a geometry that facilitates grasping of the elongate body 51 during attachment/removal of the sampling device (not shown), such as via a pulling, a squeezing, and/or a threading of the sampling device. The elongate body 51 may have a first end 52a that may include a connector 53a that may selectively hold the outer shell of the sampling device to permit removal and transfer of the sampling device. The tool 50 may further include a second end 52b that may include a connector 53b that may selectively hold an outer shell of another sampling device to permit removal and transfer of the other sampling device. For example, the tool 50 may hold an unused sampling device at one of the first and second ends 52a, 52b and may hold a used sampling device at the other one of the first and second ends 52a, 52b.

The connectors 53a, 53b may each include at least one of threads, tapers, snaps, splines, and O-rings that may be adapted to connect to complimentary structures of the sampling devices and that may provide a holding force sufficient to support the weight of the sampling devices. Accordingly, the connectors 53a, 53b may be selectively snap-fit, press-fit, and/or threaded to the complimentary structures of the sampling devices to realize removable connections thereto.

One or both of the first and second ends 52a, 52b of the tool 50 may include a respective guard 54a, 54b removably connected to the elongate body 51. The guards 54a, 54b may be movable along a longitudinal axis of the elongate body 51 between respective stowed positions in which the guards 54a, 54b are retracted inwardly from the respective first and second ends 52a, 52b of the tool 50 and respective extended positions in which the guards 54a, 54b extend outwardly beyond the respective first and second ends 52a, 52b of the tool 50 to protect the sampling device(s) that may be held by the tool 50. One or both of the guards 54a, 54b may include a respective spring 55a, 55b that biases the respective guards 54a, 54b towards the extended position.

As shown in FIGS. 9-10C, one or both of the guards 54a, 54b may be provided with a respective handle 57a, 57b that may be slideably disposed within a respective groove 56a, 56b of the elongate body 51. Accordingly, a user may slide one or both of the handles 57a, 57b within the respective grooves 56a, 56b to move the respective guards 54a, 54b between the stowed and extended positions.

FIGS. 11-12C illustrate a third exemplary embodiment of a sampling device 300 that serves as a micro-aspiration device. The sampling device 300 includes a depressor 326 adapted to selectively draw the sample into and expel the sample from an opening 323 of the sampling device 300. The sampling device 300 may be adapted to collect a fixed volume of sample and/or a user determined volume of sample. In addition, the sampling device 300 may be adapted to collect the sample at a fixed shear rate and/or at a negative pressure. FIG. 11 shows a schematic cross-sectional view of the sampling device 300. FIG. 12A shows a perspective view of the sampling device 300. FIG. 12B shows a perspective view of the sampling device 300 with the depressor 326 in a depressed position. FIG. 12C shows a perspective view of the sampling device 300 having a guard 350 in a deployed position.

As illustrated in FIGS. 11-12C, the sampling device 300 may include an outer shell 310, which may be an elongate tube. The sampling device 300 may have an interior 312 and a first open end 314a. The outer shell 310 may include a geometry that facilitates grasping of the outer shell 310 during attachment/removal of the sampling device 300, such as via a pulling, a squeezing, and/or a threading of the sampling device 300. The first open end 314a of the outer shell 310 may include a connector 315 that may be provided on an interior of the first open end 314a. The connector 315 may selectively seal the first open end 314a to the sample processor (not shown) and to the medical device (not shown). The connector 315 may also removably connect a sample collector 320 (described below) of the sampling device 300 to the medical device to collect the sample and may removably connect the sample collector 320 to the sample processor to transfer the collected sample to the sample processor. The connector 315 may include, for example, at least one of male/female luer fittings, threads, tapers, snaps, splines, and O-rings adapted to connect to complimentary structures of the medical device and the sample processor. Accordingly, the connector 315 may be selectively snap-fit, press-fit, and/or threaded to the complimentary structures of the medical device and the sample processor to realize removable connections thereto. The removable connections between the connector 315 and the medical device/sample processor may form a seal. According to aspects of the invention, the sampling device 300 may be removably connected to and provide a seal with the medical device without disrupting the function of the medical device (i.e., without reducing flow, without forming air pockets, and/or without generating interrupted surfaces within the medical devices), which reduces the risk of thrombus formation in the patient treated by the medical device and/or the development of unintended bacterial growth.

The sample collector 320 that may be at least partially disposed within the interior 312 of the outer shell 310 and may collect the sample from the medical device. The sample collector 320 may include an opening 323 provided at a first end 322a of the sample collector 320, which may communicate with and collect the sample from an interior of the medical device and which may further communicate with and deposit the collected sample into the sample processor. The sample collector 320 may also include an internal sample chamber C that may receive the sample, and a depressor 326 that may change an internal volume of the internal sample chamber C to selectively draw the sample into and expel the sample from the internal sample chamber C. The internal sample chamber C may be comprised of hollow interiors of the first end 322a and the depressor 326 of the sample collector 320, and at least a portion of the interior 312 of the outer shell 310.

The depressor 326 may be a flexible material that may be biased in a convex shape. The depressor 326 may further be biased in an initial position via a spring 327; the initial position may maximize a range of depression of the depressor 326. In addition, the opening 323 of the sample collector 320 may be provided at the first open end 314a of the outer shell 310. The depressor 326 may be provided at a second end 322b of the sample collector 320 that opposes the opening 323 along a longitudinal axis of the outer shell 310 and that may be located at a second open end 314b of the outer shell 310.

The sampling device 300 may include a guard 350 movably connected to the outer shell 310. The guard 350 may be movable along a longitudinal axis of the outer shell 310 between a stowed position in which the guard 350 is retracted inwardly away from the first open end 314a of the outer shell 310 and an extended position in which the guard 350 extends outwardly beyond the first open end 314a of the outer shell 310 to protect the sample collector 320. The guard 350 may include a spring 352 that biases the guard 350 towards the extended position.

The sampling device 300 may be directly connected to at least one access port of the medical device (not shown) to permit fluid communication between the sample collector 320 and an interior of the medical device. Alternatively, the sampling device 300 may be connected to the interposed port (as described above) or to any exposed portion of the medical device that is external to the patient. The connector 315 may be connected to the medical device and may hold the opening 323 of the sample collector 320 in a position tangential to adjacent interior surfaces (not shown) of the medical device such that the opening 323 of the sample collector 320 and the adjacent interior surfaces of the medical device together provide a continuous interior surface (not shown) within the medical device. That is, the opening 323 of the sample collector 320 may be adapted so as to not interrupt an internal geometry of the medical device when the sampling device 300 is connected to the medical device. According to aspects of the invention, the continuous interior surface provided by the opening 323 of the sample collector 320 and the adjacent interior surfaces of the medical device prevents the formation of interrupted surfaces within the interior of medical device, which may form a habitus for unintended bacterial growth.

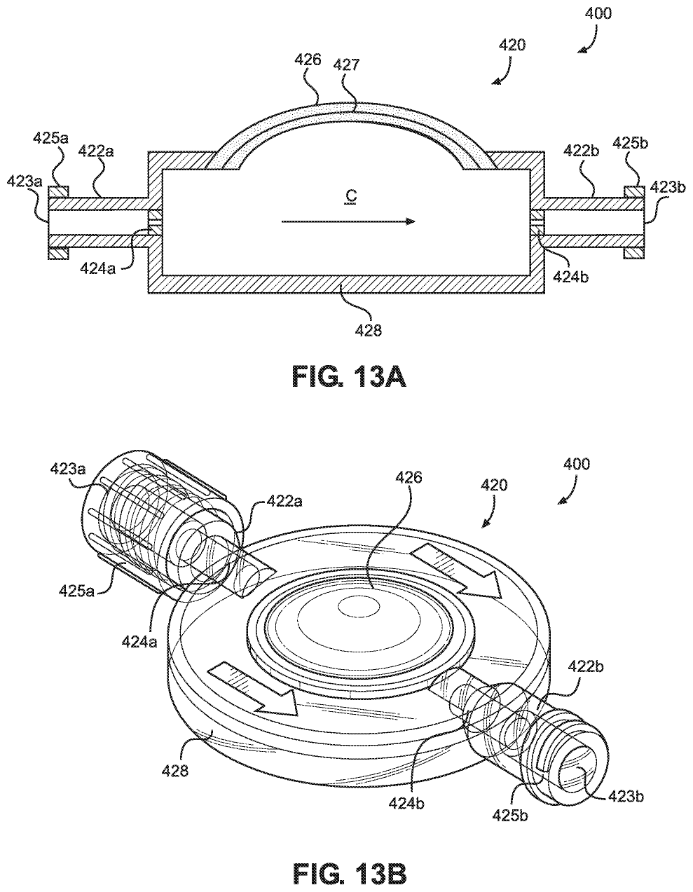

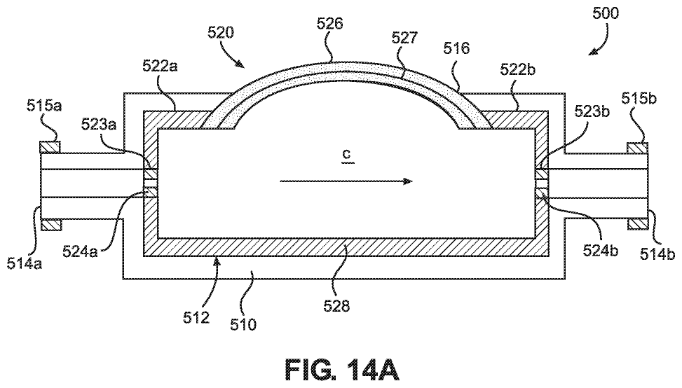

FIGS. 13A and 13B illustrate a fourth exemplary embodiment of a sampling device 400 that is adapted to pump the sample through the sampling device 400. The sampling device 400 may include a depressor 426 adapted to selectively draw the sample through an opening 423a of a first end 422a of the sampling device 400 and to expel the sample from an opening 423b of a second end 422b of the sampling device 400. The sampling device 400 may also be adapted to collect a fixed volume of sample and/or a user determined volume of sample. In addition, the sampling device 400 may be adapted to collect the sample at a fixed shear rate and/or at a negative pressure. FIG. 13A shows a schematic cross-sectional view of the sampling device 400. FIG. 13B shows a perspective view of the sampling device 400.

As shown in FIGS. 13A and 13B, the sampling device 400 may include a sample collector 420 that may collect a sample from the medical device (not shown). The sample collector 420 may include a first end 422a that may have an opening 423a that may communicate with and collect the sample from an interior of the medical device. The sample collector 420 may further include a second end 422b that opposes the first end 422a. The second end 422b may have an opening 423b that may communicate with and transfer the sample to the sample processor (not shown). The sample collector 420 may further include an internal sample chamber C that may receive the sample, and a depressor 426 that may selectively change an internal volume of the internal sample chamber C to selectively draw the sample into and expel the sample from the internal sample chamber C. The internal sample chamber C may be comprised of hollow interiors of the first end 422a, the second end 422b, a central portion 428 arranged between the first end 422a and the second end 422b, and the depressor 426 of the sample collector 420. The opening 423a of the first end 422a of the sample collector 420 may be removably connected to the medical device to collect the sample and the opening 423b of the second end 422b of the sample collector 420 may be removably connected to the sample processor to transfer the sample to the sample processor.

The depressor 426 may be a flexible material that may be biased in a convex shape. The depressor 426 may further be biased in an initial position via a spring 427; the initial position may maximize a range of depression of the depressor 426. The depressor 426 may be provided at the central portion 428 of the sample collector 420.

The opening 423a of the first end 422a of the sample collector 420 and the opening 423b of the second end 422b of the sample collector 420 may each include a respective one way valve 424a, 424b. The one way valves 424a, 424b may be oriented to limit flow of the sample in one direction along a path from the opening 423a of the first end 422a of the sample collector 420, through the internal sample chamber C, and out the opening 423b of the second end 422b of the sample collector 420. A depression/pump of the depressor 426 may pump the sample through the path. A single pump of the depressor 426 may collect and/or expel a predetermined volume of sample. The sample collector 420 may be comprised of a transparent material to permit monitoring of sample flow through the internal sample chamber C.

The first end 422a of the sample collector 420 may include a connector 425a that may removably connect the sample collector 420 to the medical device to collect the sample. The second end 422b of the sample collector 420 may also include a connector 425b; the connector 425b of the second end 422b may removably connect the sample collector 420 to the sample processor to transfer the sample from the sample collector 420 to the sample processor. The connectors 425a, 425b may each include, for example, at least one of male/female luer fittings, threads, tapers, snaps, splines, and O-rings adapted to connect to complimentary structures of the medical device and the sample processor. Accordingly, the connectors 425a, 425b may be selectively snap-fit, press-fit, and/or threaded to the complimentary structures of at least one of the medical device and the sample collector to realize removable connections thereto. The removable connection between the connectors 425a, 425b and the respective medical device/ sample processor may form a seal. According to aspects of the invention, the sampling device 400 may be removably connected to and provide a seal with the medical device without disrupting the function of the medical device (i.e., without reducing flow, without forming air pockets, and/or without generating interrupted surfaces within the medical devices), which reduces the risk of thrombus formation in the patient treated by the medical device and/or the development of unintended bacterial growth.

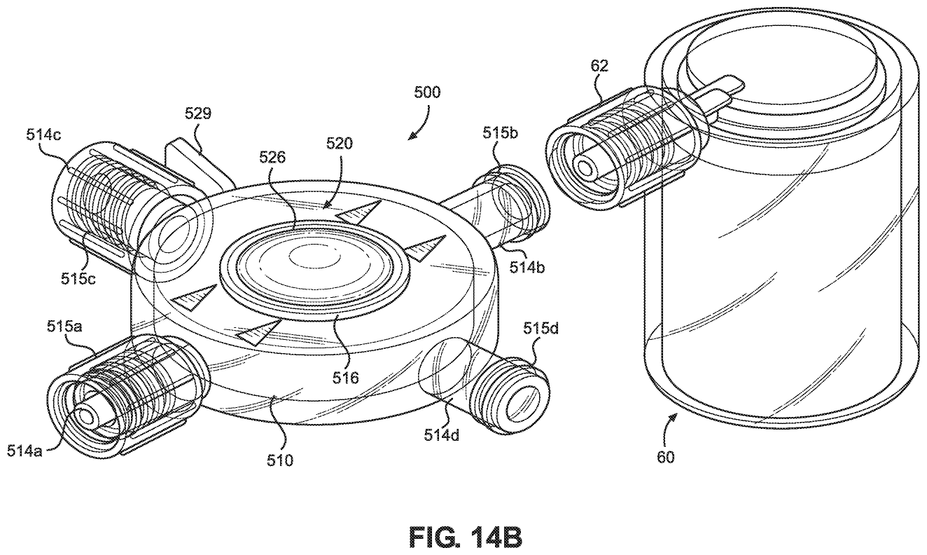

FIGS. 14A and 14B illustrate a fifth exemplary embodiment of a sampling device 500, which is also adapted to pump the sample through the sampling device 500. The sampling device 500 may include a sample collector 520 that may be rotatably held within an outer shell 510. The sampling device 500 may be adapted to collect a fixed volume of sample and/or a user determined volume of sample. In addition, the sampling device 500 may be adapted to collect the sample at a fixed shear rate and/or at a negative pressure. FIG. 14A shows a schematic cross-sectional view of the sampling device 500. FIG. 14B shows a perspective view of the sampling device 500 disconnected from a waste container 60 of the infection detection system 1.

As shown in FIGS. 14A and 14B, the sampling device 500 may include a sample collector 520 that may collect a sample from the medical device (not shown). The sample collector 520 may include a first end 522a that may have an opening 523a that may communicate with and collect the sample from an interior of the medical device. The sample collector 520 may further include a second end 522b that opposes the first end 522a. The second end 522b may have an opening 523b that may communicate with and transfer the sample to the sample processor (not shown). The sample collector 520 may further include an internal sample chamber C that may receive the sample, and a depressor 526 that may selectively change an internal volume of the internal sample chamber C to selectively draw the sample into and expel the sample from the internal sample chamber C. The internal sample chamber C may be comprised of hollow interiors of the first end 522a, the second end 522b, a central portion 528 arranged between the first end 522a and the second end 522b, and the depressor 526 of the sample collector 520. The opening 523a of the first end 522a of the sample collector 520 may be removably connected to the medical device to collect the sample and the opening 523b of the second end 522b of the sample collector 520 may be removably connected to the sample processor to transfer the sample to the sample processor.

The depressor 526 may be a flexible material that may be biased in a convex shape. The depressor 526 may further be biased in an initial position via a spring 527; the initial position may maximize a range of depression of the depressor 526. The depressor 526 may be provided at the central portion 528 of the sample collector 520.

The opening 523a of the first end 522a of the sample collector 520 and the opening 523b of the second end 522b of the sample collector 520 may each include a respective one way valve 524a, 524b. The one way valves 524a, 524b may be oriented to limit flow of the sample in one direction along a path from the opening 523a of the first end 522a of the sample collector 520, through the internal sample chamber C, and out the opening 523b of the second end 522b of the sample collector 520. A depression/pump of the depressor 526 may pump the sample through the path. A single pump of the depressor 526 may collect and/or expel a predetermined volume of sample. The sample collector 520 may be comprised of a transparent material to permit monitoring of sample flow through the internal sample chamber C.

The sampling device 500 may further include an outer shell 510 having an interior 512. The sample collector 520 may be provided within the interior 512 of the outer shell 510. The sample collector 520 may be rotatable within the interior 512 of the outer shell 510. The outer shell 510 may include a first window 516 provided through the outer shell 510. The first window 516 of the outer shell 510 may have a perimeter that may accommodate the depressor 526 therein. The first window 516 may be aligned with the depressor 526 such that the depressor 526 may be depressed without interference from the outer shell 510. The outer shell 510 may include a second window (not shown) that may be provided through a portion of a perimeter of the outer shell 510. The sample collector 520 may include a handle 529 that may extend through the second window of the outer shell 510 and that may move freely within the second window upon a rotation of the sample collector 520. The handle 529 may be utilized by a user to initiate the rotation of the sample collector 520 within the outer shell 510. Both the sample collector 520 and the outer shell 510 may be comprised of a transparent material to permit monitoring of sample flow through the internal sample chamber C.

The outer shell 510 may further include a first open end 514a that may have a connector 515a. The connector 515a may be removably connected to the medical device to allow fluid communication between the first open end 514a and the medical device. The outer shell 510 may further include a second open end 514b that may have a connector 515b. The connector 515b may be removably connected to the sample processor to allow fluid communication between the second open end 514b and the sample processor. The outer shell 510 may further include a third open end 514c that may have a connector 515c. The connector 515c may be removably connected to the medical device to allow fluid communication between the third open end 514c and the medical device. The outer shell 510 may further include a fourth open end 514d that may have a connector 515d. The connector 515d may be removably connected to a connector 62 of a waste container 60 to allow fluid communication between the fourth open end 514d and the waste container 60. The waste container 60 may be filed with an absorbent material (e.g., gauze) that may hold and trap waste materials. According to aspects of the invention, waste material (such as catheter lock solution) may be pumped through the sample collector 520 prior to the sample collection to reduce dilution of the later collected sample.

The connectors 515a, 515b, 515c, 515d may each include, for example, at least one of male/female luer fittings, threads, tapers, snaps, splines, and O-rings adapted to connect to complimentary structures of the medical device, the sample collector, and/or the waste container 60. Accordingly, the connectors 515a, 515b, 515c, 515d may be selectively snap-fit, press-fit, and/or threaded to the complimentary structures of at least one of the medical device, the sample collector, and the waste container 60 to realize removable connections thereto. At least one of the connectors 515a, 515b, 515c, 515d may be different than at least one of the other connectors 515a, 515b, 515c, 515d such that a user may selectively connect the sampling device 500 to accessories (e.g., the medical device, the sample processor, and/or the waste container 60) having a variety of different complimentary connecting structures.

The removable connection between the connectors 515a, 515b, 515c, 515d and the respective medical device/sample processor/waste container 60 may form a seal. According to aspects of the invention, the sampling device 500 may be removably connected to and provide a seal with the medical device without disrupting the function of the medical device (i.e., without reducing flow, without forming air pockets, and/or without generating interrupted surfaces within the medical devices), which reduces the risk of thrombus formation in the patient treated by the medical device and/or the development of unintended bacterial growth.

The sample collector 520 may be rotatable within the outer shell 510 between a first position and a second position. In the first position, the opening 523a of the first end 522a of the sample collector 520 may be coaxially aligned and in fluid communication with the first open end 514a of the outer shell 510. Further, in the first position the opening 523b of the second end 522b of the sample collector 520 may be coaxially aligned and in fluid communication with the second open end 514b of the outer shell 510. Accordingly, when the sample collector 520 is rotated to the first position and the depressor 526 is pumped, the sample may flow from the first open end 514a of the outer shell 510 through the sample collector 520 and out the second open end 514b of the outer shell 510.

In the second position, the opening 523a of the first end 522a of the sample collector 520 may be coaxially aligned and in fluid communication with the third open end 514c of the outer shell 510. Further, in the second position the opening 523b of the second end 522b of the sample collector 520 may be coaxially aligned and in fluid communication with the fourth open end 514d of the outer shell 510. Accordingly, when the sample collector 520 is rotated to the second position and the depressor 526 is pumped, the sample may flow from the third open end 514c of the outer shell 510 through the sample collector 520 and out the fourth open end 514d of the outer shell 510.

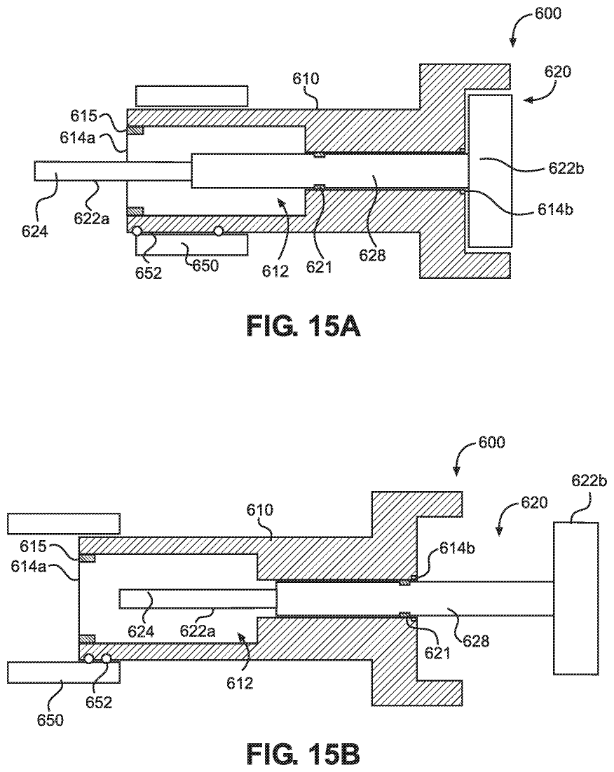

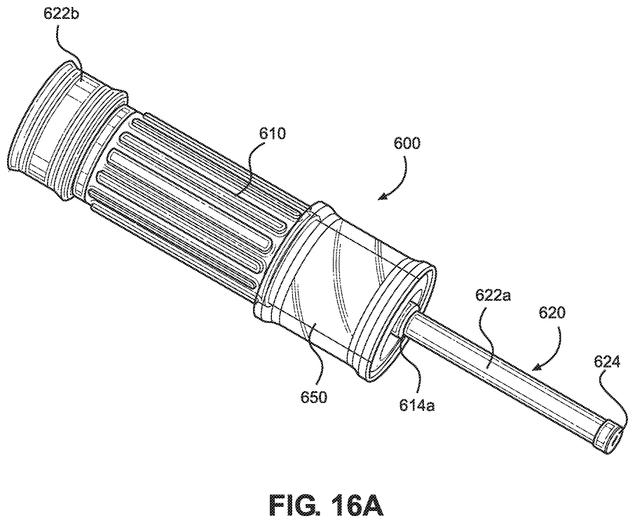

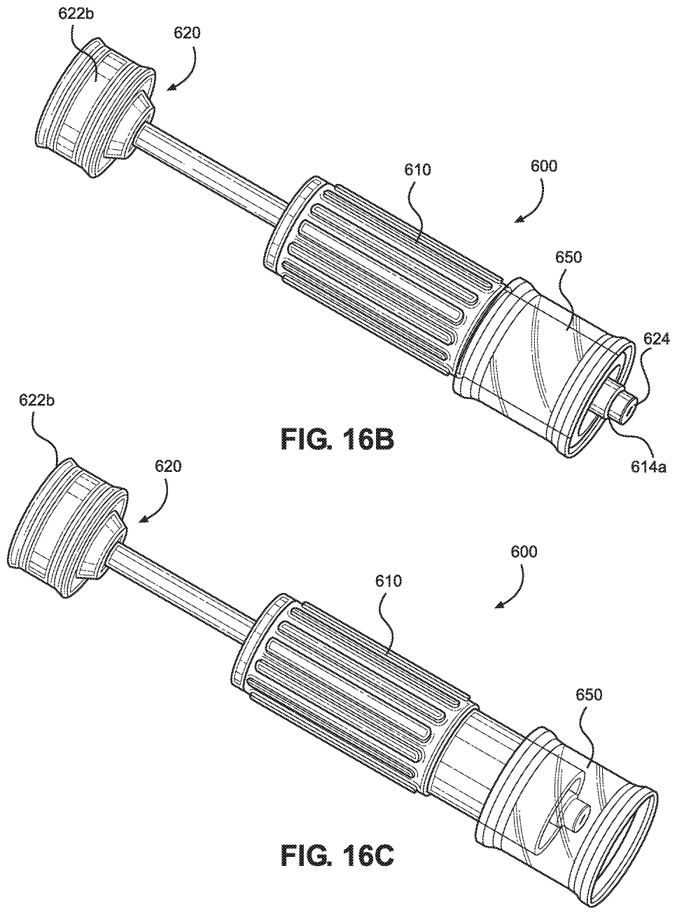

FIGS. 15A-16C illustrate a sixth exemplary embodiment of a sampling device 600 that is adapted to collect the sample by being placed in direct contact with a sample source (e.g., urine, fecal matter, purulence/pus, a suspected infection site, such as a surgical dressing, wound, and/or an insertion site, etc.) and to deposit the collected sample in the sample processor for processing. FIG. 15A shows a schematic cross-sectional view of the sampling device 600 in an extended position for collecting of the sample and/or for depositing the sample into the sample processor. FIG. 15B shows a schematic cross-sectional view of the sampling device 600 in a retracted position. FIG. 16A shows a perspective view of the sampling device 600 in the extended position. FIG. 16B shows a perspective view of the sampling device 600 in the retracted position. FIG. 16C shows a perspective view of the sampling device 600 in the retracted position with a guard 650 of the sampling device 600 in a deployed position.

As shown in FIGS. 15A and 15B, the sampling device 600 may include an outer shell 610 the may have an interior 612 and a first open end 614a. The outer shell 610 may include a geometry that facilitates grasping of the outer shell 610 during an attachment and/or a removal of the sampling device 600, such as via a pulling, a squeezing, and/or a threading of the sampling device 600. The first open end 614a may include a connector 615 that may selectively provide a seal between the first open end 614a and the sample processor (not shown). The sampling device 600 may further include a sample collector 620 that may be at least partially disposed within the interior 612 of the outer shell 610. The sample collector 620 may be adapted to move along a longitudinal axis of the outer shell 610 to an extended position, as shown in FIG. 15A, which may be used for collecting the sample and/or for depositing the sample in the sample processor. As shown in FIG. 15B, the sample collector 620 may also be adapted to move along the longitudinal axis of the outer shell 610 to a retracted position, which may be used for protecting the sample collector 620 prior to and/or after sample collection.

The sample collector 620 may include a first end 622a that may extend a predetermined distance beyond the first open end 614a of the outer shell 610 when the sample collector 620 is moved to the extended position during sample collection, as shown in FIG. 15A. For example, during sample collection the first open end 614a of the outer shell 610 may be brought into a position near, but not touching, the sample. The sample collector 620 may then be extended the predetermined distance beyond the first open end 614a to the extended position where the first end 622a may directly contact and collect the sample. The first end 622a of the sample collector 620 may be a solid shaft that may have one or more cross-sectional profiles (e.g., round, flat, threaded, star-shaped, etc.).

In the retracted position, as shown in FIG. 15B, at least a portion of the first end 622a of the sample collector 620 may be retracted within the interior 612 of the outer shell 610. Additionally, the first end 622a of the sample collector 620 may include an outer diameter and the first open end 614a of the outer shell 610 may include an inner diameter. The inner diameter of the first open end 614a of the outer shell 610 may be at least twice as large as the outer diameter of the first end 622a of the sample collector 620 to avoid exposing the collected sample to the inner diameter of the first open end 614a of the outer shell 610 in the retracted position.

The first end 622a of the sample collector 620 may include a surface 624 adapted to specifically or non-specifically bind pathogens thereto. The surface 624 may be comprised of an immobilization of an antibody (e.g., anti-Staphylococcus aureus, anti-Salmonella, and/or anti Shigella antibody), a segment of antibody that attracts pathogens, and/or a moiety that binds to a surface receptor of a pathogen (e.g., mannose binding lectin, RNAs, DNAs, and ss-DNAs). The surface 624 may additionally or alternatively be comprised of an absorbent material (e.g., a swab or sponge). The surface 624 may additionally or alternatively be adapted to limit absorption of substances other than pathogens. For example, the surface 624 may include a non-fouling background (e.g., a non-fouling polymer with functional groups for specific modification) that reduces nonspecific protein binding, thrombus formation, and/or mammalian cell attachment. The surface 624 may additionally or alternatively include at least one of pores, through holes, and structured surfaces (e.g., on the micro or nano level) that increase an effective surface area of the surface 624 and to which the sample may adhere/absorb.

The sample collector 620 may further include a second end 622b that may be arranged opposite the first end 622a in the longitudinal axis of the outer shell 610. The sample collector 620 may also include a central portion 628 arranged between the first end 622a and the second end 622b. The outer shell 610 may include a second open end 614b and the central portion 628 of the sample collector 620 may be slideably arranged within the second open end 614b of the outer shell 610. For example, an outer surface of the central portion 628 of the sample collector 620 may slide along an inner surface of the interior 612 of the outer shell 610. The second end 622b of the sample collector 620 may be a handle/knob that may control movement of the first end 622a of the sample collector 620 along the longitudinal axis of the outer shell 610 between the extended and retracted positions. The second end 622b of the sample collector 620 may also be adapted to abut the second open end 614b of the outer shell 610 to define a maximum extension length of the first end 622a of the sample collector 620. The central portion 628 of the sample collector 620 may include a seal 621 that may, for example, prevent the sample collector 620 from being completely removed from the second open end 614b of the outer shell 610. The seal 621 may abut against a portion of the second open end 614b of the outer shell 610 when the sample collector is retracted a predetermined maximum retraction distance.

The connector 615 may include, for example, at least one of male/female luer fittings, threads, tapers, snaps, splines, and O-rings adapted to connect to a complimentary structure of, e.g., the sample processor. Accordingly, the connector 615 may be selectively snap-fit, press-fit, and/or threaded to the complimentary structure of the sample processor to realize a removable connection thereto. The removable connection between the connector 615 and the sample processor may form a seal. The connector 615 may also be adapted such that after sample collection, the first end 622a of the sample collector 620 may be locked within the interior 612 of the outer shell 610 until such time as the sampling device 600 is connected to the sample processor. That is, the sampling device 600 may be adapted such that, after sample collection and a subsequent retraction of the sample collector 620, only upon a connection of the sampling device 600 to the sample processor may the sample collector 620 be released from a locked position within the interior 612 of the outer shell 610. After being released from the locked position, the first end 622a of the sample collector 620 may freely extend from the first open end 614a of the outer shell 610 to deposit the sample in the sample processor for processing.

The sampling device 600 may further include a guard 650 movably connected to the outer shell 610. The guard 650 may be movable along a longitudinal axis of the outer shell 610 between a stowed position, as shown in FIGS. 15A, 16A, and 16B, in which the guard 650 is retracted inwardly away from the first open end 614a of the outer shell 610. The guard 650 may also be movable along the longitudinal axis of the outer shell 610 to an extended position, as shown in FIGS. 15B and 16C, in which the guard 650 extends outwardly beyond the first open end 614a of the outer shell 610 to protect the sample collector 620. The guard 650 may include a spring 652 that biases the guard 650 towards the extended position.

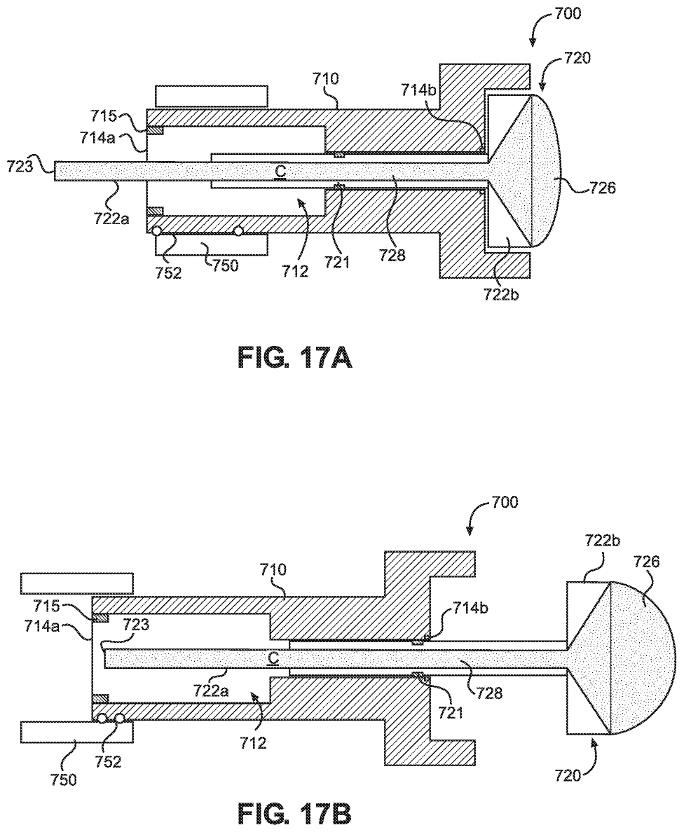

FIGS. 17A and 17B illustrate a seventh exemplary embodiment of a sampling device 700 that is adapted to draw a sample into the sampling device 700 directly from a sample source (e.g., urine, fecal matter, purulence/pus, a suspected infection site, such as a surgical dressing, wound, and/or an insertion site, etc.) and is further adapted to deposit the collected sample in the sample processor for processing. FIG. 17A shows a schematic cross-sectional view of the sampling device 700 in an extended position for collecting of the sample and/or for depositing the sample into the sample processor. FIG. 17B shows a schematic cross-sectional view of the sampling device 700 in a retracted position.

As shown in FIGS. 17A and 17B, the sampling device 700 may include an outer shell 710 the may have an interior 712 and a first open end 714a. The outer shell 710 may include a geometry that facilitates grasping of the outer shell 710 during an attachment and/or a removal of the sampling device 700, such as via a pulling, a squeezing, and/or a threading of the sampling device 700. The first open end 714a may include a connector 715 that may selectively provide a seal between the first open end 714a and the sample processor (not shown). The sampling device 700 may further include a sample collector 720 that may be at least partially disposed within the interior 712 of the outer shell 710. The sample collector 720 may be adapted to move along a longitudinal axis of the outer shell 710 between an extended position, as shown in FIG. 17A, which may be employed for collecting the sample and/or for depositing the sample in the sample processor, and between a retracted position, as shown in FIG. 17B, which may be employed for protecting the sample collector 720 prior to and/or after sample collection.

The sample collector 720 may include a first end 722a that may extend a predetermined distance beyond the first open end 714a of the outer shell 710 when the sample collector is moved to the extended position during sample collection, as shown in FIG. 17A. For example, during sample collection the first open end 714a of the outer shell 710 may be brought into a position near, but not touching, the sample. The first end 722a of the sample collector 720 may then be extended the predetermined distance beyond the first open end 714a to the extended position where the first end 722a may directly contact and collect the sample. When the sample collector 720 is moved to the retracted position, as shown in FIG. 17B, at least a portion of the first end 722a of the sample collector 720 may be retracted within the interior 712 of the outer shell 710. The first end 722a may be a hollow shaft that may have one or more cross-sectional profiles (e.g., round, flat, threaded, star-shaped, etc.). Additionally, the first end 722a of the sample collector 720 may include an outer diameter and the first open end 714a of the outer shell 710 may include an inner diameter. The inner diameter of the first open end 714a of the outer shell 710 may be at least twice as large as the outer diameter of the first end 722a of the sample collector 720 to avoid exposing the collected sample to the inner diameter of the first open end 714a of the outer shell 710 in the retracted position.

The sample collector 720 may further include a second end 722b that may be arranged opposite the first end 722a in the longitudinal axis of the outer shell 710 and may also include a central portion 728 arranged between the first end 722a and the second end 722b. The outer shell 710 may include a second open end 714b and the central portion 728 of the sample collector 720 may be slideably arranged within the second open end 714b of the outer shell 710. For example, an outer surface of the central portion 728 of the sample collector 720 may slide along an inner surface of the interior 712 of the outer shell 710. The second end 722b of the sample collector 720 may be a handle/knob for controlling movement of the first end 722a of the sample collector 720 along the longitudinal axis of the outer shell 710 between the extended and retracted positions. The second end 722b of the sample collector 720 may also be adapted to abut the second open end 714b of the outer shell 710 to define a maximum extension length of the first end 722a of the sample collector 720. The central portion 728 of the sample collector 720 may include a seal 721 that may, for example, prevent the sample collector 720 from being completely removed from the second open end 714b of the outer shell 710. The seal 721 may abut against a portion of the second open end 714b of the outer shell 710 when the sample collector is retracted a predetermined maximum retraction distance.

The sample collector 720 may further include a depressor 726 arranged at the second end 722b and an internal sample chamber C that may receive the sample therein. The depressor 726 may be comprised of a flexible material that is biased in a convex shape and that may function as a diaphragm. The internal sample chamber C may be comprised of hollow interiors of the first end 722a, the central portion 728, the second end 722b, and the depressor 726 of the sample collector 720. The first end 722a of the sample collector 720 may include an opening 723 and the sample may be selectively drawn into and expelled from the opening 723. The internal sample chamber C may translate through an entire length of the sample collector 720 from the opening 723 to the depressor 726. At least internal surfaces that define the hollow interiors of the first end 722a, the central portion 728, the second end 722b, and the depressor 726 of the sample collector 720 may be made of a non-absorbent material.

The connector 715 may include at least one of male/female luer fittings, threads, tapers, snaps, splines, and O-rings adapted to connect to a complimentary structure of, e.g., the sample processor. Accordingly, the connector 715 may be selectively snap-fit, press-fit, and/or threaded to the complimentary structure of the sample processor to realize a removable connection thereto. The removable connection between the connector 715 and the sample processor may form a seal.

The sample collector 720 may be placed in the extended position such that the opening 723 of the first end 722a of the sample collector 720 extends the predetermined distance beyond the first open end 714a of the outer shell 710. The opening 723 of the first end 722a of the sample collector 720 may then enter the sample source and upon a depression and a subsequent release of the depressor 726, an internal volume of the internal sample chamber C may be changed to selectively draw the sample into the internal sample chamber C. After obtaining the sample, the sample collector 720 may be moved to the retracted position to protect the sample from contamination or loss. The sampling device 700 may then be connected to the sample processor via the connector 715.

The connector 715 may be adapted such that after sample collection, the first end 722a of the sample collector 720 may be locked within the interior of the outer shell 710 until such time as the sampling device 700 is connected to the sample processor. That is, the sampling device 700 may be adapted such that, after sample collection and a subsequent retraction of the sample collector 720, only upon a connection of the sampling device 700 to the sample processor may the sample collector 720 be released from a locked position within the interior 712 of the outer shell 710. After being released from the locked position, the first end 722a of the sample collector 720 may freely extend from the first open end 714a of the outer shell 710 to deposit the sample in the sample processor for processing. The sample may be deposited in the sample processor via a subsequent depression of the depressor 726.

The sampling device 700 may further include a guard 750 movably connected to the outer shell 710. The guard 750 may be movable along a longitudinal axis of the outer shell 710 to a stowed position, as shown in FIG. 17A, in which the guard 750 is retracted inwardly away from the first open end 714a of the outer shell 710. The guard 750 may also be movable along the longitudinal axis of the outer shell 710 to an extended position, as shown in FIG. 17B, in which the guard 750 extends outwardly beyond the first open end 714a of the outer shell 710 to protect the sample collector 720. The guard 750 may include a spring 752 that biases the guard 750 towards the extended position.

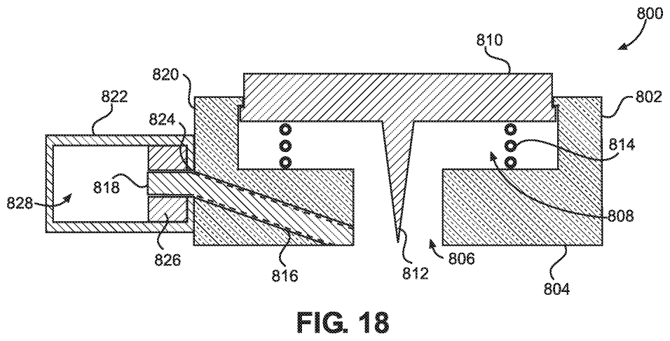

FIG. 18 illustrates an eighth exemplary embodiment of the sampling device 800 in accordance with aspects of the invention. The sampling device 800 may include a housing 802 having a bottom surface 804 that may be placed in contact with a patient (not shown). The housing 802 may further include an opening 806 extending through the bottom surface 804. The sampling device 800 may further include a cavity 808 provided within the housing 802. The opening 806 may extend through the bottom surface 804 to the cavity 808.

The sampling device 800 may also include a plunger 810 slidably disposed within the cavity 808 of the housing 802. The plunger 810 may include a lancet 812 that may be subcutaneously injected into the patient to cause whole blood to flow from the patient and pool on a skin surface of the patient for subsequent collection. For example, the sampling device 800 may include a spring 814 disposed within the cavity 808 that biases the plunger 810 upward in an initial position at which the lancet 812 is contained within the housing 802. A user may exert a downward force on the plunger 810 to overcome the upward bias exerted by the spring 814 and to plunge the lancet 812 into the patient. Upon removal of the user-applied downward force, the upward bias exerted by the spring 814 may restore the initial position of the plunger 810/lancet 812 to allow whole blood to pool on the surface of the skin at the site of the subcutaneous injection.

The sampling device 800 may further include a microfluidic passage 816 extending through the housing 802. The microfluidic passage 816 may extend from the opening 806 of the housing 802 to an outlet 818 that projects from an exterior surface 820 of the housing 802 and that defines a male connector. The microfluidic passage 816 may passively draw the whole blood sample pooled on the surface of the skin from the opening 806 and out the outlet 818. For example, the microfluidic passage 816 may be shaped to induce capillary flow of the whole blood sample through the microfluidic passage 816. Additionally and/or alternatively, the sampling device 800 may be arranged to utilize gravitational forces to induce flow of the whole blood sample through the microfluidic passage 816. In embodiments, the sampling device 800 may include a pump (not shown) that may pump the whole blood sample through the microfluidic passage 816. Further, the microfluidic passage 816 and/or the outlet 818 may be shaped to limit or terminate flow of the whole blood sample after a predetermined amount of whole blood has flowed through the microfluidic passage 816 and out the outlet 818. For example, the microfluidic passage 816 may be designed to utilize surface tension affects to limit the flow of the whole blood sample. Accordingly, the whole blood sample may be collected by the sampling device 800 (e.g., as a result of the design of the microfluidic passage 816) in a metered, predetermined quantity.

The sampling device 800 may also include a detachable reservoir 822. The reservoir 822 may include an opening 824 that may define a female connector. The opening 824 of the reservoir 822 may be removably connected to the outlet 818 of the housing 802. For example, the opening 824 of the reservoir 822 may be press-fit or threaded to the outlet 818 of the housing 802. The reservoir 822 may further include a seal 826 that may automatically seal the opening 824 upon a detachment of the reservoir 822. For example, the seal 826 may automatically seal the opening 824 upon removal of the reservoir 822 from the outlet 818 of the housing 802 and/or from the sample processor 900. The automatic seal 826 may be structurally similar to self-sealing aspects of needleless connectors, as would be readily understood by a person having ordinary skill in the art. The reservoir 822 may further include a cavity 828 that may receive and contain the whole blood sample discharged from the outlet 818. The reservoir 822 may include substantially transparent portions and may further include markings to indicate that a sufficient quantity of the whole blood sample has been collected. The reservoir 822 may further include a diaphragm (not shown) and/or may be made of a resiliently flexible material such the that the whole blood sample may be pumped out of the reservoir 822 upon connection to the sample processor 900.

The sampling device 800 may be sterilized prior to sample collection and may be stored in a sealed, sterile packaging (not shown). Further, interior surfaces (e.g., of the microfluidic passage 816 and/or the reservoir 822) of the sampling device 800 that may contact the whole blood sample may be pre-coated with an anticoagulant, such as heparin, to automatically prevent or reduce coagulation of the whole blood sample prior to sample processing. Accordingly, the whole blood sample collected by the sampling device 800 may be automatically exposed to anticoagulant in the sampling device 800 and may be ready for processing at the sample processor 900 without necessitating additional treatment/intervention by a user/clinician.

The sampling device 800 depicted in FIG. 18 and described above outlines features of a whole blood sampling device 800 in accordance with aspects of the invention. Further features of the invention that may additionally and/or alternatively be included in the sampling device 800, including but not limited to features of the microfluidic passage 816 and/or of components to effectuate the subcutaneous injection, are described in U.S. Pat. Nos. 9,289,763, 9,033,898, and 8,808,202 and in U.S. patent application Ser. Nos. 14/932,485, 14/816,994, and 15/387,177, which are respectively published as U.S. Pub. Nos. 2016/0051981, 2016/0174888, and 2017/0172481. Each of U.S. Pat. Nos. 9,289,763, 9,033,898, and 8,808,202 and of U.S. patent application Ser. Nos. 14/932,485, 14/816,994, and 15/387,177 are hereby incorporated by reference in their entirety.

FIG. 19 illustrates a schematic exemplary embodiment of the sample processor 900 in accordance with aspects of the invention. The sample processor 900 may include a plurality of chambers, for example, a first chamber 902, a second chamber 904, and/or a third chamber 906. The first chamber 902 may include a lysate, such as, a lyophilized Acris lysing chemistry. The lysate contained within the first chamber 902 may be in the form of a reagent plug(s) having dried lysis reagents. The first chamber 902 may be placed in fluid communication with the reservoir 822 after the reservoir 822 is removed from the outlet 818 of the sampling device 800. Accordingly, the whole blood sample contained within the reservoir 822 may be transferred to the first chamber 902 during processing. The sample processor 900 may include a male connector (not shown) that is complementary (e.g., for a press-fit/threaded fit) with the female connector formed by the opening 824 of the reservoir 822. Accordingly, the male connector may effectuate the fluid communication between the opening 824 of the reservoir 822 and the first chamber 902 of the sample processor 900. The male connector may automatically release the seal 826 of the reservoir 822 to open the reservoir 822 and permit transfer of the whole blood sample.

The second chamber 904 may include a diluent and may be in fluid communication with the first chamber 902. The diluent may flow from the second chamber 904 to the first chamber 902 to form a template for processing. The template may contain the lysate, the diluent, and the whole blood sample. The diluent may be communicated to the first chamber 902 in advance of the arrival of the whole blood sample to prepare the lysate. Alternatively, the diluent and the whole blood sample may arrive at the first chamber 902 simultaneously. The third chamber 906 may include another diluent. For example, the third chamber 906 may include an NASBA diluent.

The sample processor 900 may further include a plurality of reaction tubes 908-938. The reaction tubes 908-938 may each include at least one window that may allow for inspection of reactions that may occur within the reaction tubes 908-938. Each of the reaction tubes 908-938 may contain regents, at least one of which may be unique to each individual reaction tube. The reagents may induce targeted NASBA reactions for determining the presence of a particular pathogen in a provided sample. Accordingly, the sample processor 900 may process as many unique targeted NASBA reactions on the sample as there are reaction tubes in the sample processor 900. Each of the plurality of reaction tubes 908-938 may be in fluid communication with the first chamber 902, the second chamber 904, and/or the third chamber 906 via conduits (not shown). For example, the NASBA diluent contained within the third chamber 906 may be communicated to each of the plurality of reaction tubes 908-938 a predetermined period (e.g., 5 minutes) before introduction of the template. After expiration of the predetermined period, the template containing the lysate, the diluent, and the whole blood may be distributed to each of the plurality of reaction tubes 908-938 to induce the NASBA reactions and the results of the NASBA reactions may be analyzed in the analytical instrument 30.

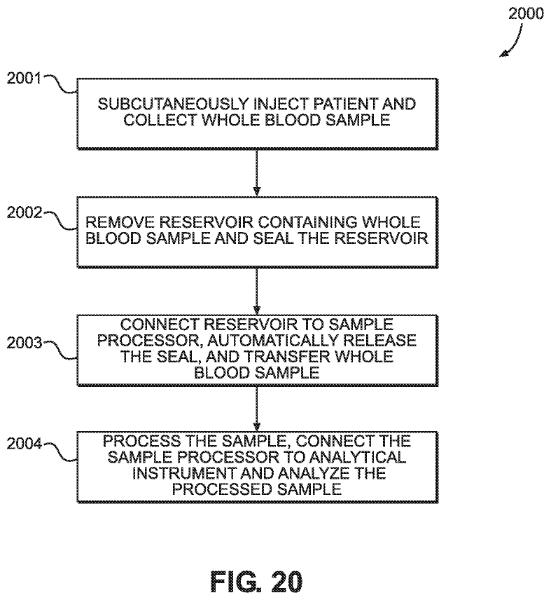

FIG. 20 illustrates an exemplary process 2000 for detecting an infection in a patient, which may be executed using the infection detection system 1 in accordance with the above-described embodiments. In particular, the exemplary process 2000 may be executed using an infection detection system 1 with the sampling device 800 and the sample processor 900. The process 2000 may include, at a first step 2001, subcutaneously injecting the patient with the lancet 812 of the sampling device 800 and collecting a whole blood sample in the reservoir 822. For example, a user may exert a downward force on the plunger 810 of the sampling device 800 and thereby plunge the lancet 812 into the patient. Upon removal of the user-applied downward force, an upward bias exerted by the spring 814 of the sampling device 800 may restore the initial position of the plunger 810/lancet 812 to allow whole blood to pool on the surface of the skin at the site of the subcutaneous injection.

The whole blood pooled on the surface of the skin may be passively drawn (e.g., via a capillary force) through the microfluidic passage 816 of the sampling device, out the outlet 818, and into the reservoir 822. The process 2000 may further include automatically exposing the whole blood sample to an anticoagulant that is coated onto interior surfaces (e.g., of the microfluidic passage 816 and/or the reservoir 822) of the sampling device 800 to automatically prevent or reduce coagulation of the whole blood sample prior to sample processing. The anticoagulant may be, for example, heparin. The anticoagulant may be coated during manufacturing of the sampling device 800, i.e., prior to collection of the whole blood sample. Accordingly, the whole blood sample collected by the sampling device 800 may be automatically exposed to anticoagulant in the sampling device 800 and may be made ready for processing at the sample processor 900 without necessitating any additional treatment steps by a user/clinician.

At a second step 2002, the process 2000 may include a user removing the reservoir 822 from the sampling device 800 and the sampling device 800 may automatically and concurrently seal the reservoir 822 upon removal. For example, the seal 826 may automatically seal the opening 824 upon removal from the outlet 818 of the housing 802.

At a third step 2003, the process 2000 may further include connecting the reservoir 822 to the sample processor 900, automatically releasing the seal 826 of the reservoir 822, and transferring the whole blood sample from the reservoir 822 to the sample processor 900. According to aspects of the invention, the reservoir 822 may remain sealed from the moment that the reservoir 822 is removed from the outlet 818 of the housing 802 to the moment that the reservoir 822 is connected to the sample processor 900. Accordingly, the probability of contamination of the whole blood sample is diminished. The reservoir 822 may be directly connected to the sample processor 900 immediately upon or a short period of time after removal from the outlet 818 of the housing 802. That is, the reservoir 822 may be directly connected to the sample processor 900 after removal from the outlet 818 of the housing 802 without any intervening steps (e.g., sample treatment, storage, and/or extraneous transport) within a period of time for a typical user to traverse the distance between the sampling device 800 and the sample processor 900 to connect the reservoir 822 thereto. By directly connecting the reservoir 822 to the sample processor 900, processing of the whole blood sample may be rapidly performed (e.g., in less than 30 minutes) at the location that the sample is collected, eliminating the need for time-wasting intermediary sample treatment, storage, and/or extraneous transport of the reservoir 822.

The opening 824 of the reservoir 822 may define a female connector and the sample processor 900 may include a complimentary male connector that, when connected to the female connector, automatically releases the seal 826 of the reservoir 822 and provides a fluid connection between the reservoir 822 and the sample processor 900. The reservoir 822 may be disposed in a vertical position when connected to the sample processor 900 such that gravity may be used to urge the whole blood sample into the sample processor 900. Additionally or alternatively, the reservoir 822 may include a flexible diaphragm/material that a user may press/squeeze to urge the whole blood sample into the sample processor 900. The sample processor 900 may include a pump that automatically pumps the whole blood sample into the sample processor 900.

The process 2000 may conclude, at a fourth step 2004, by processing the whole blood sample in the sample processor 900, connecting the sample processor 900 to the analytical instrument 30, and analyzing the processed sample via the analytical instrument 30. For example, the sample processor 900 may be initialized automatically upon connection with the reservoir 822. That is, the sample processor 900 may include a system (e.g., including a controller, sensors, pumps, valves, etc.) that may detect that the reservoir 822 is connected and automatically control the processing of the sample upon detection. Alternatively, the sample processor 900 may be initialized upon insertion into the analytical instrument 30 and/or upon user interaction with a human machine interface of the analytical instrument 30. For example, the analytical instrument 30 may automatically control seals and/or may pump fluids from any of the reservoir 822, the first chamber 902, the second chamber 904, and the third chamber 906 to control the processing of the whole blood sample.

Processing of the whole blood sample may include mixing the diluent contained within the second chamber 904 with the lysate contained within the first chamber 902. Subsequently or simultaneously, the whole blood sample may be mixed with the lysate and diluent to form a whole blood template for lysing pathogen cells and extracting and purifying pathogen messenger RNA (i.e., dissolve targeted mRNA and remove inhibitors that could interfere with nucleic acid amplification). While the whole blood sample, the lysate, and the diluent are mixed, the process 2000 may include fluidly communicating the diluent from the third chamber 906 to the plurality of reaction tubes 908-938 and thus concurrently hydrating all of the reagents/structures/materials disposed in the plurality of reaction tubes 908-938. The whole blood sample, lysate, and diluent may be mixed in the first chamber 902 while the reagents/structures/materials disposed in the plurality of reaction tubes 908-938 are separately hydrated with diluent for a predetermined period of time (e.g., 5 minutes). Upon expiration of the predetermined period of time, the whole blood sample, lysate, diluent mix may be fluidly communicated to each of the plurality of reaction tubes 908-938 for, e.g., isothermal amplification of targeted mRNA to identify the presence of specific genes within the whole blood template.

According to aspects of the invention, the entirety of the sample processing may occur within the various components of the infection detection system 1 thereby obviating the need of direct user interaction with the sample to effectuate sample processing. For example, the whole blood sample may be spiked with anticoagulant within the sampling device 800, as discussed above. The whole blood sample may remain sealed within the reservoir 822 while transferred to the sample processor 900, reducing the risk of contamination or of exposing the user of the infection detection system to pathogens that may be within the whole blood sample. The infection detection system 1 may accordingly be used by a user of low skill and may be readily transported to and applied in a variety of environments (e.g., the home, a hospital room, etc.). Further, the entire process 2000 may be performed in proximity to the patient. Proximity to the patient may mean in a single facility and/or room where the patient is disposed, such as in a home, a hospital, an ambulance, etc. As a result, infection in a patient may be rapidly detected and identified, which may improve the prognosis of the patient.

The many features and advantages of the infection detection system and methods described herein are apparent from the detailed specification, and thus, the claims cover all such features and advantages within the scope of this application. Further, numerous modifications and variations are possible. As such, it is not desired to limit the infection detection system to the exact construction and operation described and illustrated and, accordingly, all suitable modifications and equivalents may fall within the scope of the claims.

1. A method of detecting an infection in a patient using an infection detection system comprising a sampling device, a sample processor, and an analytical instrument, the method comprising:
collecting a whole blood sample in a reservoir of the sampling device;
connecting the reservoir to the sample processor and transferring the whole blood sample from the reservoir to the sample processor;
processing the whole blood sample in the sample processor to produce a processed sample;
connecting the sample processor to the analytical instrument and analyzing the processed sample via the analytical instrument, comprising performing a nucleic acid sequence-based amplification (NASBA)-based nucleic-acid assay for mRNA and/or DNA on the processed sample.

2. The method according to claim 1, wherein the method is performed in its entirety in proximity to the patient.

3. The method according to claim 2, wherein the method is performed in its entirety in a single facility.

4. The method according to claim 2, wherein the method is performed in its entirety in a single room.

5. The method according to claim 1, wherein the whole blood sample is subcutaneously collected from the patient.

6. The method according to claim 1, wherein subcutaneously collecting the whole blood sample includes passively drawing the whole blood sample through a microfluidic passage of the sampling device.

7. The method according to claim 1, further comprising removing the reservoir and concurrently sealing the reservoir.

8. The method according to claim 7, wherein the connecting the reservoir to the sample processor includes automatically terminating the sealing of the reservoir.

9. The method according to claim 1, further comprising automatically exposing the whole blood sample to an anticoagulant that is coated onto an interior surface of the sampling device.

10. The method according to claim 9, wherein the interior surface of the sampling device includes at least one of an interior surface of the reservoir and an interior surface of a microfluidic passage of the sampling device.

11. The method according to claim 1, wherein processing the whole blood sample in the sample processor comprises mixing lysate, a first diluent, and the whole blood sample in a first chamber.

12. The method according to claim 11, wherein processing the whole blood sample in the sample processor comprises hydrating reagents contained within at least one reaction tube of the sample processor with a second diluent.

13. The method according to claim 12, wherein mixing the lysate, the first diluent, and the whole blood sample in the first chamber occurs concurrently with the hydrating of the reagents contained within the at least one reaction tube.

14. The method according to claim 13, wherein mixing the lysate, the first diluent, and the whole blood sample in the first chamber and the hydrating of the reagents contained within the at least one reaction tube occurs for a predetermined period of time.

15. The method according to claim 14, wherein processing the whole blood sample in the sample processor further comprises supplying the lysate, the first diluent, and the whole blood sample to the at least one reaction tube upon expiration of the predetermined period of time.

16. The method according to claim 1, wherein the collecting of the whole blood sample in the reservoir includes metering the whole blood sample to collect a predetermined quantity of the whole blood sample.
